(19) United States
(12) Patent Application Publication
(10) Pub. No.: US 2025/0302715 A1
(43) Pub. Date: Oct. 2, 2025

(54) COMPOSITION COMPRISING N,N-DICARBOXYMETHYLGLUTAMIC ACID, AT LEAST ONE FATTY ALCOHOL, AT LEAST ONE FATTY ACID, AT LEAST ONE POLYOL, AT LEAST ONE ALKALINE AGENT AND OPTIONALLY AT LEAST ONE DYE

(71) Applicant: L'Oreal, Paris (FR)

(72) Inventors: Sarah Boulemnakher, Saint Ouen (FR); Hanène Moueddene, Saint Ouen (FR); Julie Bruyere, Saint Ouen (FR); Mickaël Agach, Saint Ouen (FR)

(73) Assignee: L'Oreal, Paris (FR)

(21) Appl. No.: 18/573,900

(22) PCT Filed: Jun. 29, 2022
(86) PCT No.: PCT/EP2022/067998

§ 371 Date: Dec. 22, 2023

(30) Foreign Application Priority Data
Jun. 30, 2021 (FR) 2107101

(51) Int. Cl. A61K 8/44; A61Q 5/06; A61Q 5/08; A61Q 5/10

(52) U.S. Cl. CPC A61K 8/44; A61Q 5/065; A61Q 5/08; A61Q 5/10; A61K 2800/4322; A61K 2800/4324; A61K 2800/5922; A61K 2800/882

(57) ABSTRACT

The present invention relates to a cosmetic composition for treating keratin fibres, notably human keratin fibres such as the hair, comprising N,N-dicarboxymethylglutamic acid, a salt thereof, solvates thereof and/or solvates of the salts thereof, at least one fatty alcohol, at least one fatty acid, at least one polyol and at least one alkaline agent, in a total content of greater than or equal to 5% by weight, relative to the total weight of the composition, and optionally at least one dye.

The present invention also relates to a process for dyeing and/or lightening keratin fibres, such as the hair, in which the composition as described previously, and optionally comprising at least one dye chosen from oxidation dyes, direct dyes and mixtures thereof, is applied to said fibres.

The present invention also relates to the use of the composition according to the invention for dyeing and/or lightening keratin fibres such as the hair.

The present invention relates to a cosmetic composition for treating keratin fibres, notably human keratin fibres such as the hair, comprising N,N-dicarboxymethylglutamic acid, a salt thereof, solvates thereof and/or solvates of the salts thereof, at least one fatty alcohol, at least one fatty acid, at least one polyol and at least one alkaline agent, in a total content of greater than or equal to 5% by weight, relative to the total weight of the composition, and optionally at least one dye.

The present invention also relates to a process for dyeing and/or lightening keratin fibres, such as the hair, in which the composition as described previously, and optionally comprising at least one dye chosen from oxidation dyes, direct dyes and mixtures thereof, is applied to said fibres.

The present invention also relates to the use of the composition according to the invention for dyeing and/or lightening keratin fibres such as the hair.

In processes for dyeing keratin fibres, it is known practice to dye keratin fibres via various techniques using direct dyes for non-permanent dyeing, or oxidation dye precursors for permanent dyeing.

Non-permanent dyeing or direct dyeing consists in dyeing keratin fibres with dye compositions containing direct dyes. These dyes are coloured and colouring molecules that have affinity for keratin fibres. They are applied to the keratin fibres for a time necessary to obtain the desired colouring, and are then rinsed out.

Some of these dyes may be used under lightening conditions, which enables the production of colourings that are visible on dark hair.

It is also known practice to dye keratin fibres permanently via oxidation dyeing. This dyeing technique consists in applying to the keratin fibres a composition containing dye precursors such as oxidation bases and couplers. Under the action of an oxidizing agent, these precursors form one or more coloured substances in the hair.

The existing dyeing and/or lightening processes generally use cosmetic compositions comprising a certain number of ingredients for which the regulatory conditions are increasingly stringent. It is thus necessary to develop compositions comprising alternative ingredients.

Consumers are notably in search of dyeing and/or lightening products that are more environmentally friendly, notably based on ingredients of natural origin, and which have good working qualities, are easy to use and give good dyeing and/or lightening properties.

Furthermore, the “natural” dye compositions that are currently available on the market do not make it possible to obtain colourings that are sufficiently persistent with respect to external agents such as light, perspiration, washing, or even rubbing.

The performance of the colourings obtained with such compositions is also poorer compared with the performance obtained with conventional dye compositions, notably in terms of colour build-up, strength and chromaticity.

Thus, there is a real need for a composition for dyeing and/or lightening keratin fibres, in particular human keratin fibres such as the hair, which is more environmentally friendly and which does not have the abovementioned drawbacks, i.e. which is capable of giving good performance, notably in terms of colour build-up, strength and chromaticity, when it comprises one or more dyes, and/or good lightening performance while at the same time having low selectivity and good persistence and which is capable of giving good dyeing performance, and/or good lightening performance, even after a period of storage, while at the same time having good working qualities.

One subject of the present invention is thus a composition comprising:
one or more compounds chosen from N,N-dicarboxymethylglutamic acid, salts thereof, solvates thereof, solvates of the salts thereof and mixtures thereof,
one or more fatty alcohols,
one or more fatty acids,
one or more polyols,
one or more alkaline agents in a total content of greater than or equal to 5% by weight relative to the total weight of the composition, and
optionally one or more dyes.

The composition according to the invention makes it possible to achieve the above objectives, notably in terms of colour build-up, dyeing power, chromaticity, selectivity, persistence of the colouring, in particular with respect to shampoos when it comprises one or more dyes, and/or good performance in terms of lightening, and also good working qualities.

The composition according to the invention also makes it possible to obtain powerful colourings and to lead to good cosmeticity, notably in terms of sheen, a more natural feel, and a good level of comfort, while at the same time preserving the integrity of the fibre.

The composition according to the invention can also lead to good lightening performance.

The composition according to the invention also has the advantage of leading to efficient lightening of keratin fibres while at the same time reducing the unpleasant and aggressive odours, notably of ammonia, which are liable to be given off when it is applied to said fibres.

Moreover, the composition according to the invention shows good stability, notably on storage. The composition may notably comprise dyes and/or other compounds in salt form.

Moreover, the composition according to the invention has good working qualities, notably a creamy texture allowing quick and easy mixing with an oxidizing composition, where appropriate, and easy and uniform spreading over the entire head of hair. The composition according to the invention has good stability over time, notably little or no change in its viscosity during storage.

The present invention also relates to a process for dyeing and/or lightening keratin fibres, in particular human keratin fibres such as the hair, in which a composition is applied to said fibres, comprising:
one or more compounds chosen from N,N-dicarboxymethylglutamic acid, salts thereof, solvates thereof, solvates of the salts thereof and mixtures thereof,
one or more fatty alcohols,
one or more fatty acids,
one or more polyols,
one or more alkaline agents in a total content of greater than or equal to 5% by weight relative to the total weight of the composition, and
optionally one or more dyes.

In particular, the present invention also relates to a process for dyeing keratin fibres, in particular human keratin fibres such as the hair, in which a composition is applied to said fibres, comprising:
one or more compounds chosen from N,N-dicarboxymethylglutamic acid, salts thereof, solvates thereof, solvates of the salts thereof and mixtures thereof,
one or more fatty alcohols,
one or more fatty acids,
one or more polyols,
one or more alkaline agents in a total content of greater than or equal to 5% by weight relative to the total weight of the composition, and
one or more dyes.

The process according to the invention can also lead to good lightening performance.

The present invention also relates to the use of the composition according to the invention for dyeing and/or lightening keratin fibres, preferably dyeing keratin fibres, in particular human keratin fibres such as the hair.

Other subjects, characteristics, aspects and advantages of the invention will emerge even more clearly on reading the description and the example that follows.

In the text hereinbelow, unless otherwise indicated, the limits of a range of values are included in that range, notably in the expressions “between” and “ranging from . . . to . . . ”.

Moreover, the expression “at least one” used in the present description is equivalent to the expression “one or more”.

### N,N-Dicarboxymethylglutamic Acid, Salts Thereof

The composition according to the invention comprises one or more compounds chosen from N,N-dicarboxymethylglutamic acid, salts thereof, solvates thereof, solvates of the salts thereof and mixtures thereof.

The salts are notably alkali metal, alkaline-earth metal, ammonium and substituted ammonium salts.

Among the salts of these compounds, the alkali metal salts and notably the sodium or potassium salts are preferred.

The composition according to the invention preferably comprises tetrasodium glutamate diacetate (GLDA). Use will be made, for example, of Dissolvine GL38 or 45S from AkzoNobel.

Preferably, the total content of compound(s) chosen from N,N-dicarboxymethylglutamic acid, salts thereof, solvates thereof, solvates of the salts thereof and mixtures thereof ranges from 0.001% to 15% by weight, more preferentially from 0.005% to 10% by weight, better still from 0.01% to 8% by weight, even better still from 0.05% to 5% by weight, or even from 0.075% to 2% by weight, or even from 0.1% to 1% by weight, relative to the total weight of the composition.

According to a preferred embodiment, the total content of tetrasodium glutamate diacetate (GLDA) ranges from 0.001% to 15% by weight, more preferentially from 0.005% to 10% by weight, better still from 0.01% to 8% by weight, even better still from 0.05% to 5% by weight, or even from 0.075% to 2% by weight, or even from 0.1% to 1% by weight, relative to the total weight of the composition.

### Fatty Alcohols

The composition according to the invention comprises one or more fatty alcohols preferably chosen from C₁₄-C₄₀ fatty alcohols.

For the purposes of the present invention, the term “fatty alcohol” means a long-chain aliphatic alcohol comprising from 6 to 40 carbon atoms, preferably from 8 to 30 carbon atoms, and comprising at least one hydroxyl group OH.

For the purposes of the present invention, the fatty alcohols are neither oxyalkylenated nor glycerolated.

In particular, the fatty alcohols are different from nonionic surfactants.

Preferably, the fatty alcohols according to the invention are neither (poly)oxyalkylenated or (poly)glycerolated and are different from nonionic surfactants.

The fatty alcohols according to the invention may be saturated or unsaturated, and linear or branched, and include from 8 to 40 carbon atoms.

More preferentially, the fatty alcohols according to the invention are chosen from compounds having the structure R—OH with R denoting a linear or branched, saturated or unsaturated alkyl group optionally substituted with one or more hydroxyl groups, comprising from 8 to 40, better still from 10 to 30, or even from 12 to 24 and even better still from 14 to 22 carbon atoms.

The fatty alcohols may be chosen from liquid fatty alcohols and solid fatty alcohols, and mixtures thereof.

For the purposes of the present invention, the term “solid fatty alcohol” means a fatty alcohol with a melting point of greater than 25° C., preferably greater than or equal to 28° C., more preferentially greater than or equal to 30° C. at atmospheric pressure (1.013×10⁵ Pa).

The solid fatty alcohols may be chosen from saturated or unsaturated, linear or branched solid fatty alcohols, including from 8 to 40 carbon atoms.

The solid fatty alcohols that may be used according to the invention are preferably chosen from compounds having the structure R—OH with R denoting a saturated linear alkyl group, optionally substituted with one or more hydroxyl groups, comprising from 8 to 40, better still from 10 to 30, or even from 12 to 24 and even better still from 14 to 22 carbon atoms.

The solid fatty alcohols that may be used may be chosen, alone or as a mixture, from:
lauryl alcohol (or 1-dodecanol);
myristyl alcohol (or 1-tetradecanol);
cetyl alcohol (or 1-hexadecanol);
stearyl alcohol (or 1-octadecanol);
arachidyl alcohol (or 1-eicosanol);
behenyl alcohol (or 1-docosanol);
lignoceryl alcohol (or 1-tetracosanol);
ceryl alcohol (or 1-hexacosanol);
montanyl alcohol (or 1-octacosanol);
myricyl alcohol (or 1-triacontanol).

Among the mixtures of solid fatty alcohols, use will preferably be made of cetyl alcohol, stearyl alcohol and/or mixtures thereof such as cetearyl alcohol.

For the purposes of the present invention, the term “liquid fatty alcohol” means a fatty alcohol with a melting point of less than or equal to 25° C., preferably less than or equal to 20° C. at atmospheric pressure (1.013×10⁵ Pa).

The liquid fatty alcohols that may be used according to the invention are preferably chosen from compounds having the structure R—OH with R denoting a saturated or unsaturated, linear or branched, preferably unsaturated and/or branched, alkyl group optionally substituted with one or more hydroxyl groups, comprising from 8 to 40, better still from 10 to 30, or even from 12 to 24 and even better still from 14 to 22 carbon atoms.

Preferably, the liquid fatty alcohols are chosen from compounds having the structure R—OH with R denoting an unsaturated and/or branched, preferably unsaturated, alkyl group optionally substituted with one or more hydroxyl groups, comprising from 12 to 24 and better still from 14 to 22 carbon atoms.

The liquid fatty alcohols that may be used may be chosen, alone or as a mixture, from oleyl alcohol, linoleyl alcohol, linolenyl alcohol, isocetyl alcohol, isostearyl alcohol, 2-octyl-1-dodecanol, 2-butyloctanol, 2-hexyl-1-decanol, 2-decyl-1-tetradecanol and 2-tetradecyl-1-cetanol, and mixtures thereof; use will preferably be made of oleyl alcohol.

Preferably, the liquid fatty alcohols are chosen from linoleyl alcohol, oleyl alcohol, linolenyl alcohol and mixtures thereof, preferably oleyl alcohol.

Preferably, the fatty alcohol(s) are chosen from fatty alcohols comprising 14 to 40 carbon atoms, more preferentially 14 to 22 carbon atoms, better still cetyl alcohol, stearyl alcohol, cetearyl alcohol, oleyl alcohol and mixtures thereof.

According to a preferred embodiment, the composition according to the invention comprises one or more solid fatty alcohols and one or more liquid fatty alcohols, preferably one or more solid fatty alcohols comprising 14 to 40 carbon atoms, more preferentially 14 to 22 carbon atoms and one or more liquid fatty alcohols comprising 14 to 40 carbon atoms, more preferentially 14 to 22 carbon atoms.

According to a preferred embodiment, the composition according to the invention comprises one or more solid fatty alcohols comprising from 14 to 22 carbon atoms and one or more liquid fatty alcohols comprising from 14 to 22 carbon atoms.

According to another preferred embodiment, the composition according to the invention comprises one or more solid fatty alcohols chosen from compounds having the structure R—OH with R denoting a linear saturated alkyl group, optionally substituted with one or more hydroxy groups, comprising from 14 to 22 carbon atoms, and one or more liquid fatty alcohols chosen from compounds having the structure R—OH with R denoting an unsaturated and/or branched, preferentially unsaturated, alkyl group, optionally substituted with one or more hydroxyl groups, comprising from 14 to 22 carbon atoms.

Advantageously, the fatty alcohol(s) are present in a total content ranging from 1% to 35% by weight, preferably from 5% to 30% by weight, more preferentially from 8% to 25% by weight, and better still from 10% to 20% by weight, relative to the total weight of the composition.

According to a particular embodiment, advantageously, the solid fatty alcohol(s) are present in a total content ranging from 1% to 35% by weight, preferably from 5% to 30% by weight, more preferentially from 8% to 25% by weight and better still from 10% to 20% by weight, relative to the total weight of the composition.

According to another particular embodiment, the liquid fatty alcohol(s) are advantageously present in a total content ranging from 0.5% to 15% by weight, more preferentially from 1% to 10% by weight and better still from 2% to 5% by weight relative to the total weight of the composition.

### Fatty Acid

The composition according to the invention also comprises one or more fatty acids.

The term “fatty acid” means a long-chain carboxylic acid comprising at least 6 carbon atoms, in particular from 6 to 40 carbon atoms, preferably from 8 to 30 carbon atoms. The fatty acids according to the invention preferentially comprise from 10 to 30 carbon atoms and better still from 14 to 22 carbon atoms. They may optionally be hydroxylated.

In particular, fatty acids in free form, for instance fatty acids that are not in the form of esters (or fatty acids that are not esterified).

Preferably, the fatty acids present in the composition according to the invention include at least one carboxylic acid group and a linear or branched, saturated or unsaturated, in particular unsaturated, alkyl chain comprising from 6 to 40 carbon atoms, preferably from 8 to 30 carbon atoms, more preferentially from 10 to 30 carbon atoms and better still from 14 to 22 carbon atoms.

Preferably, the fatty acids include at least one carboxylic acid group and a linear or branched, saturated or unsaturated, in particular unsaturated, alkyl chain comprising from 10 to 30 carbon atoms, in particular from 14 to 22 carbon atoms.

Preferably, the fatty acids include at least one carboxylic acid group and a linear or branched, unsaturated alkyl chain comprising from 14 to 22 carbon atoms.

More preferentially, the fatty acids according to the invention are chosen from compounds having the structure R—C(O)OH in which R represents a linear or branched, saturated or unsaturated alkyl group comprising from 6 to 40 carbon atoms, preferably from 8 to 30 carbon atoms, preferentially from 12 to 24 carbon atoms, better still from 14 to 20 carbon atoms.

For the purposes of the present invention, the fatty acids present in the composition are neither oxyalkylenated nor glycerolated.

The fatty acids may be chosen from solid fatty acids and liquid fatty acids, and mixtures thereof.

For the purposes of the present invention, the term “solid fatty acid” means a fatty acid with a melting point of greater than 25° C., preferably greater than or equal to 28° C., more preferentially greater than or equal to 30° C. at atmospheric pressure (1.013×10⁵ Pa).

The solid fatty acids used in the present invention are notably chosen from myristic acid, cetylic acid, stearylic acid, palmitic acid, arachidic acid, stearic acid, lauric acid, behenic acid, 12-hydroxystearic acid, and mixtures thereof.

Particularly preferably, the solid fatty acid(s) are chosen from lauric acid, myristic acid, cetylic acid and stearic acid.

For the purposes of the present invention, the term “liquid fatty acid” means a fatty acid with a melting point of less than or equal to 25° C., preferably less than or equal to 20° C. at atmospheric pressure (1.013×10⁵ Pa).

The liquid fatty acid(s) according to the invention may be chosen from oleic acid, linoleic acid, arachidonic acid, isostearic acid, isopalmitic acid, and mixtures thereof, preferentially oleic acid.

Preferably, the fatty acids present in the composition according to the invention are liquid fatty acids, in particular chosen from fatty acids including at least one carboxylic acid group and a linear or branched, unsaturated alkyl chain comprising from 10 to 30 carbon atoms, in particular from 14 to 22 carbon atoms.

Preferably, the fatty acids present in the composition according to the invention are liquid fatty acids, in particular chosen from oleic acid, linoleic acid, linolenic acid and mixtures thereof, more preferentially oleic acid.

Preferably, the fatty acids present in the composition according to the invention are lauric acid, oleic acid, linoleic acid, linolenic acid, undecylenic acid, isocetylic acid, isostearic acid, cetylic acid, stearic acid and cetylstearic acid, and mixtures thereof, preferably oleic acid.

Preferably, the fatty acid(s) are chosen from myristic acid, palmitic acid, stearic acid, oleic acid, and mixtures thereof, preferentially oleic acid.

Advantageously, the fatty acid(s) are present in a total content ranging from 0.1% to 15% by weight, preferably from 0.5% to 10% and preferentially from 1% to 5% by weight, relative to the total weight of the composition.

In a particular embodiment, the liquid fatty acid(s) are advantageously present in a total content ranging from 0.1% to 15% by weight, preferably from 0.5% to 10% and preferentially from 1% to 5% by weight relative to the total weight of the composition.

### Polyols

The composition according to the invention also comprises one or more polyols.

For the purposes of the present invention, the term “polyol” means an organic compound constituted of a hydrocarbon-based chain optionally interrupted with one or more oxygen atoms and bearing at least two free hydroxyl groups (—OH) borne by different carbon atoms, this compound possibly being cyclic or acyclic, linear or branched, and saturated or unsaturated.

Preferably, the polyols are different from the fatty alcohols as defined previously.

The polyols are namely different from nonionic surfactants.

More particularly, the polyol(s) comprise from 2 to 30 hydroxyl groups, more preferentially from 2 to 10 hydroxyl groups, even more preferentially from 2 to 3 hydroxyl groups.

The polyol(s) are preferably chosen from diglycerol, glycerol, propylene glycol, propane-1,3-diol, 1,3-butylene glycol, pentane-1,2-diol, octane-1,2-diol, dipropylene glycol, hexylene glycol, ethylene glycol, polyethylene glycols, sorbitol, sugars, such as glucose, and mixtures thereof, preferably from glycerol, propane-1,3-diol and mixtures thereof.

Preferably, the composition comprises one or more polyols chosen from diglycerol, glycerol, propylene glycol, propane-1,3-diol, 1,3-butylene glycol, pentane-1,2-diol, octane-1,2-diol, dipropylene glycol, hexylene glycol, ethylene glycol, polyethylene glycols, sorbitol, sugars, such as glucose, and mixtures thereof, preferably from glycerol, propane-1,3-diol and mixtures thereof.

Preferably, the composition comprises one or more polyols chosen from propane-1,3-diol, glycerol and mixtures thereof.

Advantageously, the polyol(s) are present in a total content of greater than or equal to 5% by weight relative to the total weight of the composition.

More advantageously, the polyol(s) are present in a total content ranging from 1% to 20% by weight, preferentially from 3% to 10% by weight relative to the total weight of the composition.

### Alkaline Agent

The composition according to the present invention also comprises one or more alkaline agents chosen from mineral, organic or hybrid alkaline agents.

For the purposes of the present invention, the terms “alkaline agent” and “basifying agent” are used interchangeably.

The mineral basifying agent(s) are preferably chosen from aqueous ammonia, alkali metal carbonates or bicarbonates such as sodium (hydrogen) carbonate and potassium (hydrogen) carbonate, alkali metal or alkaline-earth metal phosphates such as sodium phosphates or potassium phosphates, sodium or potassium hydroxides, alkali metal or alkaline-earth metal silicates or metasilicates such as sodium metasilicate, and mixtures thereof.

The organic basifying agent(s) are preferably chosen from alkanolamines, amino acids, organic amines, oxyethylenated and/or oxypropylenated ethylenediamines, 1,3-diaminopropane, 1,3-diamino-2-propanol, spermine, spermidine and mixtures thereof.

The term “alkanolamine” means an organic amine comprising a primary, secondary or tertiary amine function, and one or more linear or branched C₁-C₈alkyl groups bearing one or more hydroxyl radicals.

Organic amines chosen from alkanolamines such as monoalkanolamines, dialkanolamines or trialkanolamines comprising one to three identical or different C₁-C₄ hydroxyalkyl radicals are in particular suitable for performing the invention.

In particular, the alkanolamine(s) are chosen from monoethanolamine (MEA), diethanolamine, triethanolamine, monoisopropanolamine, diisopropanolamine, N,N-dimethylethanolamine, 2-amino-2-methyl-1-propanol, triisopropanolamine, 2-amino-2-methyl-1,3-propanediol, 3-amino-1,2-propanediol, 3-dimethylamino-1,2-propanediol, tris(hydroxymethyl)aminomethane and mixtures thereof.

Advantageously, the amino acids are basic amino acids comprising an additional amine function. Such basic amino acids are preferably chosen from histidine, lysine, arginine, ornithine and citrulline.

The organic amine may also be chosen from organic amines of heterocyclic type. Besides histidine that has already been mentioned in the amino acids, mention may in particular be made of pyridine, piperidine, imidazole, triazole, tetrazole and benzimidazole. The organic amine may also be chosen from amino acid dipeptides. As amino acid dipeptides that may be used in the present invention, mention may notably be made of carnosine, anserine and balenine. The organic amine may also be chosen from compounds including a guanidine function. As amines of this type other than arginine that may be used in the present invention, mention may notably be made of creatine, creatinine, 1,1-dimethylguanidine, 1,1-diethylguanidine, glycocyamine, metformin, agmatine, n-amidoalanine, 3-guanidinopropionic acid, 4-guanidinobutyric acid and 2-([amino(imino)methyl]amino)ethane-1-sulfonic acid.

Use may be made in particular of guanidine carbonate or monoethanolamine hydrochloride as hybrid compounds.

The alkaline agent(s) that are useful according to the invention are preferably chosen from alkanolamines such as monoethanolamine, diethanolamine or triethanolamine; aqueous ammonia, carbonates or bicarbonates such as sodium (hydrogen) carbonate and potassium (hydrogen) carbonate, alkali metal or alkaline-earth metal silicates or metasilicates such as sodium metasilicate and mixtures thereof, more preferentially from aqueous ammonia and alkanolamines, better still from alkanolamines and even better still monoethanolamine.

Preferably, the basifying agents are organic.

In a particular embodiment, the composition according to the invention is free of aqueous ammonia.

The total content of the alkaline agent(s) is greater than or equal to 5% by weight relative to the total weight of the composition.

Preferably, the total content of alkaline agents is greater than or equal to 8% by weight, more preferentially greater than or equal to 10% by weight, relative to the total weight of the composition.

Preferably, the total content of alkaline agents ranges from 5% to 40% by weight, more preferentially from 6% to 30% by weight, better still from 8% to 20% by weight, and even better still from 10% to 15% by weight relative to the total weight of the composition.

In a preferred embodiment, the total content of the alkaline agent(s) chosen from the alkanolamines, preferably monoethanolamine, is preferably greater than or equal to 5% by weight, preferentially ranging from 5% to 40% by weight, more preferentially from 6% to 30% by weight, even more preferentially from 8% to 20% by weight, better still from 10% to 15% by weight, relative to the total weight of the composition.

According to one embodiment, the pH of the composition is between 8 and 13, preferably between 9.0 and 12.

The pH of the composition may be adjusted to the desired value by means of acidic or alkaline agent(s) commonly used in the dyeing of keratin fibres, such as those described previously, or alternatively using buffer systems known to those skilled in the art.

### The Dyes

The composition according to the present invention may comprise one or more dyes.

Preferably, the composition according to the present invention comprises one or more dyes chosen from oxidation dyes, direct dyes and mixtures thereof, more preferentially one or more dyes chosen from oxidation dyes.

### The Dyes: Oxidation Dyes

The oxidation dyes may be chosen from one or more oxidation bases, optionally in combination with one or more couplers.

Preferably, the oxidation dye(s) comprise one or more oxidation bases.

Preferably, the composition according to the invention comprises one or more oxidation bases.

The oxidation bases may be present in the form of salts, solvates and/or solvates of salts.

The addition salts of the oxidation bases present in the composition according to the invention are notably chosen from the addition salts with an acid, such as the hydrochlorides, hydrobromides, sulfates, citrates, succinates, tartrates, lactates, tosylates, benzenesulfonates, methanesulfonates, phosphates and acetates, and the addition salts with a base such as sodium hydroxide, potassium hydroxide, aqueous ammonia, amines or alkanolamines.

Moreover, the solvates of the oxidation bases more particularly represent the hydrates of said oxidation bases and/or the combination of said oxidation bases with a linear or branched C₁ to C₄ alcohol such as methanol, ethanol, isopropanol or n-propanol. Preferably, the solvates are hydrates.

The oxidation bases may be chosen from para-phenylenediamines, bis(phenyl)alkylenediamines, para-aminophenols, ortho-aminophenols and heterocyclic bases, and the addition salts thereof, the solvates thereof, and solvates of the salts thereof.

Among the para-phenylenediamines, examples that may be mentioned include para-phenylenediamine, para-tolylenediamine, 2-chloro-para-phenylenediamine, 2,3-dimethyl-para-phenylenediamine, 2,6-dimethyl-para-phenylenediamine, 2,6-diethyl-para-phenylenediamine, 2,5-dimethyl-para-phenylenediamine, N,N-dimethyl-para-phenylenediamine, N,N-diethyl-para-phenylenediamine, N,N-dipropyl-para-phenylenediamine, 4-amino-N,N-diethyl-3-methylaniline, N,N-bis(β-hydroxyethyl)-para-phenylenediamine, 4-N,N-bis(β-hydroxyethyl)amino-2-methylaniline, 4-N,N-bis(β-hydroxyethyl)amino-2-chloroaniline, 2-β-hydroxyethyl-para-phenylenediamine, 2-methoxymethyl-para-phenylenediamine, 2-γ-hydroxypropyl-para-phenylenediamine; 2-fluoro-para-phenylenediamine, 2-isopropyl-para-phenylenediamine, N-(β-hydroxypropyl)-para-phenylenediamine, 2-hydroxymethyl-para-phenylenediamine, N,N-dimethyl-3-methyl-para-phenylenediamine, N-ethyl-N-(β-hydroxyethyl)-para-phenylenediamine, N-(β,γ-dihydroxypropyl)-para-phenylenediamine, N-(4′-aminophenyl)-para-phenylenediamine, N-phenyl-para-phenylenediamine, 2-β-hydroxyethyloxy-para-phenylenediamine, 2-β-acetylaminoethyloxy-para-phenylenediamine, N-(β-methoxyethyl)-para-phenylenediamine, 4-aminophenylpyrrolidine, 2-thienyl-para-phenylenediamine, 2-β-hydroxyethylamino-5-aminotoluene and 3-hydroxy-1-(4′-aminophenyl)pyrrolidine, and the addition salts thereof, the solvates thereof, and solvates of the salts thereof.

Among the para-phenylenediamines mentioned above, para-phenylenediamine, para-tolylenediamine, 2-isopropyl-para-phenylenediamine, 2-β-hydroxyethyl-para-phenylenediamine, 2-methoxymethyl-para-phenylenediamine, 2-y-hydroxypropyl-para-phenylenediamine, 2-β-hydroxyethyloxy-para-phenylenediamine, 2,6-dimethyl-para-phenylenediamine, 2,6-diethyl-para-phenylenediamine, 2,3-dimethyl-para-phenylenediamine, N,N-bis(β-hydroxyethyl)-para-phenylenediamine, 2-chloro-para-phenylenediamine and 2-β-acetylaminoethyloxy-para-phenylenediamine, and the addition salts thereof, the solvates thereof, and solvates of the salts thereof are particularly preferred.

Among the bis(phenyl)alkylenediamines, examples that may be mentioned include N,N′-bis(β-hydroxyethyl)-N,N′-bis(4′-aminophenyl)-1,3-diaminopropanol, N,N′-bis(β-hydroxyethyl)-N,N′-bis(4′-aminophenyl)ethylenediamine, N,N′-bis(4-aminophenyl)tetramethylenediamine, N,N′-bis(β-hydroxyethyl)-N,N′-bis(4-aminophenyl)tetramethylenediamine, N,N′-bis(4-methylaminophenyl)tetramethylenediamine, N,N′-bis(ethyl)-N,N′-bis(4′-amino-3′-methylphenyl)ethylenediamine and 1,8-bis(2,5-diaminophenoxy)-3,6-dioxaoctane, and the addition salts thereof, the solvates thereof, and solvates of the salts thereof.

Among the para-aminophenols, examples that may be mentioned include para-aminophenol, 4-amino-3-methylphenol, 4-amino-3-fluorophenol, 4-amino-3-chlorophenol, 4-amino-3-hydroxymethylphenol, 4-amino-2-methylphenol, 4-amino-2-hydroxymethylphenol, 4-amino-2-methoxymethylphenol, 4-amino-2-aminomethylphenol, 4-amino-2-(β-hydroxyethylaminomethyl)phenol and 4-amino-2-fluorophenol, and the addition salts thereof, the solvates thereof, and solvates of the salts thereof.

Among the ortho-aminophenols, examples that may be mentioned include 2-aminophenol, 2-amino-5-methylphenol, 2-amino-6-methylphenol and 5-acetamido-2-aminophenol, and the addition salts thereof, solvates thereof and solvates of the salts thereof.

Among the heterocyclic bases, examples that may be mentioned include pyridine derivatives, pyrimidine derivatives and pyrazole derivatives.

Among the pyridine derivatives, mention may be made of the compounds described, for example, in patents GB 1 026 978 and GB 1 153 196, such as 2,5-diaminopyridine, 2-(4-methoxyphenyl)amino-3-aminopyridine, 3,4-diaminopyridine, and the addition salts thereof, the solvates thereof, and solvates of the salts thereof.

Other pyridine oxidation bases that are useful in the present invention are the 3-aminopyrazolo[1,5-a]pyridine oxidation bases or their addition salts described, for example, in patent application FR 2 801 308. Examples that may be mentioned include pyrazolo[1,5-a]pyrid-3-ylamine, 2-acetylaminopyrazolo[1,5-a]pyrid-3-ylamine, 2-(morpholin-4-yl)pyrazolo[1,5-a]pyrid-3-ylamine, 3-aminopyrazolo[1,5-a]pyridine-2-carboxylic acid, 2-methoxypyrazolo[1,5-a]pyrid-3-ylamine, (3-aminopyrazolo[1,5-a]pyrid-7-yl)methanol, 2-(3-aminopyrazolo[1,5-a]pyrid-5-yl)ethanol, 2-(3-aminopyrazolo[1,5-a]pyrid-7-yl)ethanol, (3-aminopyrazolo[1,5-a]pyrid-2-yl)methanol, 3,6-diaminopyrazolo[1,5-a]pyridine, 3,4-diaminopyrazolo[1,5-a]pyridine, pyrazolo[1,5-a]pyridine-3,7-diamine, 7-(morpholin-4-yl)pyrazolo[1,5-a]pyrid-3-ylamine, pyrazolo[1,5-a]pyridine-3,5-diamine, 5-(morpholin-4-yl)pyrazolo[1,5-a]pyrid-3-ylamine, 2-[(3-aminopyrazolo[1,5-a]pyrid-5-yl)(2-hydroxyethyl)amino]ethanol, 2-[(3-aminopyrazolo1[1,5-a]pyrid-7-yl)(2-hydroxyethyl)amino]ethanol, 3-aminopyrazolo[1,5-a]pyridin-5-ol, 3-aminopyrazolo[1,5-a]pyridin-4-ol, 3-aminopyrazolo[1,5-a]pyridin-6-ol, 3-aminopyrazolo[1,5-a]pyridin-7-ol and 2-(3-amino-pyrazolo[1,5-a]pyridin-2-yl)oxyethanol, and the addition salts thereof, the solvates thereof, and solvates of the salts thereof.

Among the pyrimidine derivatives, mention may be made of the compounds described, for example, in patents DE 2359399, JP 88-169571, JP 05-63124 and EP 0770375 or patent application WO 96/15765, such as 2,4,5,6-tetraaminopyrimidine, 4-hydroxy-2,5,6-triaminopyrimidine, 2-hydroxy-4,5,6-triaminopyrimidine, 2,4-dihydroxy-5,6-diaminopyrimidine, 2,5,6-triaminopyrimidine and the addition salts thereof, and the tautomeric forms thereof, when a tautomeric equilibrium exists.

Among the pyrazole derivatives that may be mentioned are the compounds described in patents DE 3843892 and DE 4133957 and patent applications WO 94/08969, WO 94/08970, FR-A-2 733 749 and DE 195 43 988, such as 4,5-diamino-1-methylpyrazole, 4,5-diamino-1-(β-hydroxyethyl)pyrazole, 3,4-diaminopyrazole, 4,5-diamino-1-(4′-chlorobenzyl)pyrazole, 4,5-diamino-1,3-dimethylpyrazole, 4,5-diamino-3-methyl-1-phenylpyrazole, 4,5-diamino-1-methyl-3-phenylpyrazole, 4-amino-1,3-dimethyl-5-hydrazinopyrazole, 1-benzyl-4,5-diamino-3-methylpyrazole, 4,5-diamino-3-tert-butyl-1-methylpyrazole, 4,5-diamino-1-tert-butyl-3-methylpyrazole, 4,5-diamino-1-(β-hydroxyethyl)-3-methylpyrazole, 4,5-diamino-1-ethyl-3-methylpyrazole, 4,5-diamino-1-ethyl-3-(4′-methoxyphenyl)pyrazole, 4,5-diamino-1-ethyl-3-hydroxymethylpyrazole, 4,5-diamino-3-hydroxymethyl-1-methylpyrazole, 4,5-diamino-3-hydroxymethyl-1-isopropylpyrazole, 4,5-diamino-3-methyl-1-isopropylpyrazole, 4-amino-5-(2′-aminoethyl)amino-1,3-dimethylpyrazole, 3,4,5-triaminopyrazole, 1-methyl-3,4,5-triaminopyrazole, 3,5-diamino-1-methyl-4-methylaminopyrazole and 3,5-diamino-4-(β-hydroxyethyl)amino-1-methylpyrazole, and the addition salts thereof, the solvates thereof, and solvates of the salts thereof. Use may also be made of 4,5-diamino-1-(β-methoxyethyl)pyrazole.

Use will preferably be made of a 4,5-diaminopyrazole and even more preferentially of 4,5-diamino-1-(β-hydroxyethyl)pyrazole and/or a salt thereof, solvates thereof, and solvates of the salts thereof.

Pyrazole derivatives that may also be mentioned include diamino-N,N-dihydropyrazolopyrazolones and notably those described in patent application FR-A-2 886 136, such as the following compounds and the addition salts thereof: 2,3-diamino-6,7-dihydro-1H,5H-pyrazolo[1,2-a]pyrazol-1-one, 2-amino-3-ethylamino-6,7-dihydro-1H,5H-pyrazolo[1,2-a]pyrazol-1-one, 2-amino-3-isopropylamino-6,7-dihydro-1H,5H-pyrazolo[1,2-a]pyrazol-1-one, 2-amino-3-(pyrrolidin-1-yl)-6,7-dihydro-1H,5H-pyrazolo[1,2-a]pyrazol-1-one, 4,5-diamino-1,2-dimethyl-1,2-dihydropyrazol-3-one, 4,5-diamino-1,2-diethyl-1,2-dihydropyrazol-3-one, 4,5-diamino-1,2-bis(2-hydroxyethyl)-1,2-dihydropyrazol-3-one, 2-amino-3-(2-hydroxyethyl)amino-6,7-dihydro-1H,5H-pyrazolo[1,2-a]pyrazol-1-one, 2-amino-3-dimethylamino-6,7-dihydro-1H,5H-pyrazolo[1,2-a]pyrazol-1-one, 2,3-diamino-5,6,7,8-tetrahydro-1H,6H-pyridazino[1,2-a]pyrazol-1-one, 4-amino-i,2-diethyl-5-(pyrrolidin-1-yl)-1,2-dihydropyrazol-3-one, 4-amino-5-(3-dimethylaminopyrrolidin-1-yl)-1,2-diethyl-1,2-dihydropyrazol-3-one, 2,3-diamino-6-hydroxy-6,7-dihydro-1H,5H-pyrazolo[1,2-a]pyrazol-1-one, the salts thereof, the solvates thereof, and solvates of the salts thereof.

Use will preferably be made of 2,3-diamino-6,7-dihydro-1H,5H-pyrazolo[1,2-a]pyrazol-1-one and/or a salt thereof, a solvate thereof, or a solvate of the salts thereof.

Heterocyclic bases that will preferentially be used include 4,5-diamino-1-((3-hydroxyethyl)pyrazole and/or 2,3-diamino-6,7-dihydro-1H,5H-pyrazolo[1,2-a]pyrazol-1-one and/or 2-(3-aminopyrazolo[1,5-a]pyridin-2-yl)oxyethanol and/or a salt thereof, solvates thereof or solvates of the salts thereof.

Preferably, the oxidation base(s) are chosen from para-phenylenediamines, bis(phenyl)alkylenediamines, para-aminophenols, ortho-aminophenols, heterocyclic bases, and the corresponding addition salts, the solvates thereof and/or the solvates of the salts thereof, and mixtures thereof; more preferentially from 2-methoxymethyl-para-phenylenediamine, 2-β-hydroxyethyl-para-phenylenediamine, 2-γ-hydroxypropyl-para-phenylenediamine, and the addition salts thereof, the solvates thereof and/or the solvates of the salts thereof, and mixtures thereof.

In a particular embodiment, the composition according to the invention is free of oxidation bases chosen from para-phenylenediamine, para-toluenediamine, addition salts thereof, solvates thereof and solvates of the salts thereof.

The oxidation dye(s) may also comprise one or more couplers, which may be chosen from the couplers conventionally used for the dyeing of keratin fibres.

Preferably, the composition according to the invention comprises one or more couplers.

Preferably, the couplers are chosen from meta-phenylenediamines, meta-aminophenols, meta-diphenols, naphthalene-based couplers and heterocyclic couplers, and also the addition salts thereof, and/or the solvates thereof, and/or solvates of the salts thereof.

Examples that may be mentioned include 1,3-dihydroxybenzene, 1,3-dihydroxy-2-methylbenzene, 4-chloro-1,3-dihydroxybenzene, 1-hydroxy-3-aminobenzene, 1-methyl-2-hydroxy-4-β-hydroxyethylaminobenzene, 4-amino-2-hydroxytoluene, 5-amino-6-chloro-2-methylphenol, 2,4-diamino-1-(β-hydroxyethyloxy)benzene, 2-amino-4-(β-hydroxyethylamino)-1-methoxybenzene, 1,3-diaminobenzene, 1,3-bis(2,4-diaminophenoxy)propane, 3-ureidoaniline, 3-ureido-1-dimethylaminobenzene, sesamol, 1-β-hydroxyethylamino-3,4-methylenedioxybenzene, α-naphthol, 2-methyl-1-naphthol, 6-hydroxyindole, 4-hydroxyindole, 4-hydroxy-N-methylindole, 5-methoxy-6-hydroxyindole, 2-amino-3-hydroxypyridine, 6-hydroxybenzomorpholine, 2-amino-4-hydroxyethylaminoanisole, 3-amino-6-methoxy-2-methylaminopyridine, 3,5-diamino-2,6-dimethoxypyridine, 1-N-(β-hydroxyethyl)amino-3,4-methylenedioxybenzene, 2,6-bis(β-hydroxyethylamino)toluene, 6-hydroxyindoline, 2,6-dihydroxy-4-methylpyridine, 2-chloro-3,5-diaminopyridine, 2-chloro-3,5-diamino-6-methoxypyridine, 2-chloro-3,5-diamino-6-methylpyridine, 1-H-3-methylpyrazol-5-one, 1-phenyl-3-methylpyrazol-5-one, 4-(3,5-diaminopyridin-2-yl)-1-(2-hydroxyethyl)-1-methylpiperazin-1-ium chloride, 2,6-dimethylpyrazolo[1,5-b]-1,2,4-triazole, 2,4,6-trimethoxyaniline hydrochloride, 2,6-dimethyl[3,2-c]-1,2,4-triazole, 6-methylpyrazolo[1,5-a]benzimidazole and 2,6-diaminopyrazine, the addition salts thereof, and/or the solvates thereof, and/or solvates of the salts thereof, and mixtures thereof.

Preferably, the coupler(s) used in the invention are chosen from 1,3-dihydroxybenzene, 1,3-dihydroxy-2-methylbenzene, 4-chloro-1,3-dihydroxybenzene, 1-hydroxy-3-aminobenzene, 1-methyl-2-hydroxy-4-β-hydroxyethylaminobenzene, 4-amino-2-hydroxytoluene, 5-amino-6-chloro-2-methylphenol, 2,4-diamino-1-(β-hydroxyethyloxy)benzene, α-naphthol, 6-hydroxyindole, 2-amino-3-hydroxypyridine, 6-hydroxybenzomorpholine, 3-amino-6-methoxy-2-methylaminopyridine, 2-amino-4-hydroxyethylaminoanisole, hydroxyethyl-3,4-methylenedioxyaniline and 2-amino-5-ethylphenol, the addition salts thereof, and/or the solvates thereof, and/or solvates of the salts thereof, and mixtures thereof.

Even more preferentially, the coupler(s) used in the invention are chosen from 3-amino-6-methoxy-2-methylaminopyridine, 6-hydroxybenzomorpholine, 2,4-diamino-1-(β-hydroxyethyloxy)benzene, 2-amino-3-hydroxypyridine, 5-amino-6-chloro-2-methylphenol, 1-methyl-2-hydroxy-4-β-hydroxyethylaminobenzene, 2-amino-4-hydroxyethylaminoanisole, hydroxyethyl-3,4-methylenedioxyaniline, 2-amino-5-ethylphenol and 1-hydroxy-3-aminobenzene, the addition salts thereof, and/or the solvates thereof, and/or solvates of the salts thereof, and mixtures thereof.

In general, the addition salts of the couplers that may be used in the context of the invention are especially chosen from addition salts with an acid, such as hydrochlorides, hydrobromides, sulfates, citrates, succinates, tartrates, lactates, tosylates, benzenesulfonates, phosphates and acetates, and the addition salts with a base such as sodium hydroxide, potassium hydroxide, aqueous ammonia, amines or alkanolamines.

Moreover, the solvates more particularly represent the hydrates of these couplers and/or the combination of these couplers with a linear or branched C₁ to C₄ alcohol such as methanol, ethanol, isopropanol or n-propanol. Preferably, the solvates are hydrates.

Preferably, the oxidation dyes chosen from couplers are chosen from 6-hydroxybenzomorpholine, 2,4-diamino-1-(β-hydroxyethyloxy)benzene, 2-amino-3-hydroxypyridine, 5-amino-6-chloro-2-methylphenol, 1-methyl-2-hydroxy-4-β-hydroxyethylaminobenzene, 2-amino-4-hydroxyethylaminoanisole, hydroxyethyl-3,4-methylenedioxyaniline, 2-amino-5-ethylphenol, 1-hydroxy-3-aminobenzene, the addition salts thereof, the solvates thereof, and/or solvates of the salts thereof, and mixtures thereof.

Better still, the coupler(s) are chosen from: 6-hydroxybenzomorpholine, the addition salts thereof, the solvates thereof and/or the solvates of the salts thereof, hydroxyethyl-3,4-methylenedioxyaniline, the addition salts thereof, the solvates thereof and/or the solvates of the salts thereof, 2-amino-5-ethylphenol, the addition salts thereof, the solvates thereof and/or the solvates of the salts thereof, and mixtures thereof.

In a particular embodiment, the composition according to the invention is free of oxidation couplers chosen from resorcinol, 2-methylresorcinol, 4-chlororesorcinol, the addition salts thereof, the solvates thereof and the solvates of the salts thereof.

Advantageously, when they are present, the oxidation dyes chosen from oxidation bases, which, when they are present, are present in a total content ranging from 0.0001% to 10% by weight, preferably from 0.005% to 7% by weight and more preferentially from 0.1% to 4% by weight, relative to the total weight of the composition.

Advantageously, when they are present, the oxidation dyes chosen from couplers, which, when they are present, are present in a total content ranging from 0.0001% to 10% by weight, preferably from 0.005% to 7% by weight and more preferentially from 0.1% to 4% by weight, relative to the total weight of the composition.

Advantageously, when they are present, the oxidation dyes are present in a total content ranging from 0.0001% to 10% by weight, preferably from 0.005% to 7% by weight and more preferentially from 0.1% to 4% by weight, relative to the total weight of the composition.

### The Dyes: The Direct Dyes

The term “direct dye” means natural and/or synthetic dyes, other than oxidation dyes. These are dyes that will spread superficially on the fibre.

The synthetic direct dyes are, for example, chosen from the dyes conventionally used for direct dyeing, and among which mention may be made of all the aromatic and/or non-aromatic dyes that are commonly used, such as nitrobenzene, azo, hydrazono, nitro(hetero)aryl, tri(hetero)arylmethane, (poly)methine, carbonyl, azine, porphyrin, metalloporphyrin, quinone and in particular anthraquinone, indoamine and phthalocyanine direct dyes, and mixtures thereof.

Among the nitrobenzene direct dyes, mention may be made of: 1,4-diamino-2-nitrobenzene, 1-amino-2-nitro-4-β-hydroxyethylaminobenzene, 1-amino-2-nitro-4-bis(β-hydroxyethyl)aminobenzene, 1,4-bis(β-hydroxyethylamino)-2-nitrobenzene, 1-β-hydroxyethylamino-2-nitro-4-bis(β-hydroxyethylamino)benzene, 1-β-hydroxyethylamino-2-nitro-4-aminobenzene, 1-β-hydroxyethylamino-2-nitro-4-(ethyl)(β-hydroxyethyl)aminobenzene, 1-amino-3-methyl-4-β-hydroxyethylamino-6-nitrobenzene, 1-amino-2-nitro-4-β-hydroxyethylamino-5-chlorobenzene, 1,2-diamino-4-nitrobenzene, 1-amino-2-β-hydroxyethylamino-5-nitrobenzene, 1,2-bis(β-hydroxyethylamino)-4-nitrobenzene, 1-amino-2-tris(hydroxymethyl)methylamino-5-nitrobenzene, 1-hydroxy-2-amino-5-nitrobenzene, 1-hydroxy-2-amino-4-nitrobenzene, 1-hydroxy-3-nitro-4-aminobenzene, 1-hydroxy-2-amino-4,6-dinitrobenzene, 1-β-hydroxyethyloxy-2-β-hydroxyethylamino-5-nitrobenzene, 1-methoxy-2-β-hydroxyethylamino-5-nitrobenzene, 1-β-hydroxyethyloxy-3-methylamino-4-nitrobenzene, 1-β,γ-dihydroxypropyloxy-3-methylamino-4-nitrobenzene, 1-β-hydroxyethylamino-4-β,γ-dihydroxypropyloxy-2-nitrobenzene, 1-β,γ-dihydroxypropylamino-4-trifluoromethyl-2-nitrobenzene, 1-β-hydroxyethylamino-4-trifluoromethyl-2-nitrobenzene, 1-β-hydroxyethylamino-3-methyl-2-nitrobenzene, 1-β-aminoethylamino-5-methoxy-2-nitrobenzene, 1-hydroxy-2-chloro-6-ethylamino-4-nitrobenzene, 1-hydroxy-2-chloro-6-amino-4-nitrobenzene, 1-hydroxy-6-bis(β-hydroxyethyl)amino-3-nitrobenzene, 1-β-hydroxyethylamino-2-nitrobenzene, 1-hydroxy-4-β-hydroxyethylamino-3-nitrobenzene.

Among the azo direct dyes, mention may be made of: Basic Red 51, Basic Orange 31, Disperse Red 17, Acid Yellow 9, Acid Black 1, Basic Red 22, Basic Red 76, Basic Yellow 57, Acid Yellow 36, Acid Orange 7, Acid Red 33, Acid Red 35, Acid Yellow 23, Acid Orange 24, Disperse Black 9, Basic Brown 16, Basic Brown 17.

Among the hydrazono direct dyes, mention may be made of: Basic Yellow 87.

Among the nitroaryl direct dyes, mention may be made of: HC Blue 2, HC Yellow 2, HC Red 3,4-hydroxypropylamino-3-nitrophenol, N,N′-bis(2-hydroxyethyl)-2-nitrophenylenediamine.

Among the triarylmethane direct dyes, mention may be made of: Basic Violet 1, Basic Violet 2, Basic Violet 3, Basic Violet 4, Basic Violet 14, Basic Blue 1, Basic Blue 7, Basic Blue 26, Basic Green 1, Basic Blue 77 (also known as HC Blue 15), Acid Blue 1; Acid Blue 3; Acid Blue 7, Acid Blue 9; Acid Violet 49; Acid Green 3; Acid Green 5; Acid Green 50.

Among the quinone direct dyes, mention may be made of: Disperse Red 15, Solvent Violet 13, Acid Violet 43, Disperse Violet 1, Disperse Violet 4, Disperse Blue 1, Disperse Violet 8, Disperse Blue 3, Disperse Red 11, Acid Blue 62, Disperse Blue 7, Basic Blue 22, Disperse Violet 15, Basic Blue 99, and also the following compounds: 1-N-methylmorpholiniumpropylamino-4-hydroxyanthraquinone, 1-aminopropylamino-4-methylaminoanthraquinone, 1-aminopropylaminoanthraquinone, 5-β-hydroxyethyl-1,4-diaminoanthraquinone, 2-aminoethylaminoanthraquinone, 1,4-bis(β,γ-dihydroxypropylamino)anthraquinone, Acid Blue 25, Acid Blue 43, Acid Blue 78, Acid Blue 129, Acid Blue 138, Acid Blue 140, Acid Blue 251, Acid Green 25, Acid Green 41, Acid Violet 42, Mordant Red 3, Acid Black 48, HC Blue 16.

Among the azine direct dyes, mention may be made of: Basic Blue 17, Basic Red 2.

Among the indoamine direct dyes, mention may be made of: 2-β-hydroxyethlyamino-5-[bis(0-4′-hydroxyethyl)amino]anilino-1,4-benzoquinone, 2-β-hydroxyethylamino-5-(2′-methoxy-4′-amino)anilino-1,4-benzoquinone, 3-N-(2′-chloro-4′-hydroxy)phenylacetylamino-6-methoxy-1,4-benzoquinoneimine, 3-N-(3′-chloro-4′-methylamino)phenylureido-6-methyl-1,4-benzoquinoneimine, 3-[4′-N-(ethylcarbamylmethyl)amino]phenylureido-6-methyl-1,4-benzoquinoneimine.

The natural direct dyes are chosen, for example, from lawsone, juglone, indigo, leuco indigo, indirubin, isatin, hennotannic acid, alizarin, carthamine, morin, purpurin, carminic acid, kermesic acid, laccaic acid, purpurogallin, protocatechaldehyde, curcumin, spinulosin, apigenidin, orceins, carotenoids, betanin, chlorophylls, chlorophyllines, monascus, polyphenols or ortho-diphenols.

Among the ortho-diphenols that are useful according to the invention, mention may be made of: catechin, quercetin, brazilin, haematein, haematoxylin, chlorogenic acid, caffeic acid, gallic acid, L-DOPA, cyanidin, (−)-epicatechin, (−)-epigallocatechin, (−)-epigallocatechin 3-gallate (EGCG), isoquercetin, pomiferin, esculetin, 6,7-dihydroxy-3-(3-hydroxy-2,4-dimethoxyphenyl)coumarin, santalin A and B, mangiferin, butein, maritimetin, sulfuretin, robtein, betanidin, pericampylinone A, theaflavin, proanthocyanidin A2, proanthocyanidin B2, proanthocyanidin C1, procyanidins DP 4-8, tannic acid, purpurogallin, 5,6-dihydroxy-2-methyl-1,4-naphthoquinone, alizarin, wedelolactone and natural extracts containing same.

When the composition comprises at least one direct dye, they are preferably present in a total content ranging from 0.001% to 20% by weight, preferably from 0.005% to 15% by weight, more preferentially from 0.01% to 10% by weight, better still from 0.05% to 5%, and even better still from 0.1% to 3% by weight, relative to the weight of the composition.

Preferably, the composition according to the invention comprises one or more dyes chosen from oxidation dyes and direct dyes, more preferentially chosen from oxidation dyes.

Fatty Substances Other than Fatty Acids and Fatty Alcohols

The composition according to the invention may also comprise one or more fatty substances other than the fatty alcohols and the fatty acids as defined previously.

The term “fatty substance” means an organic compound that is insoluble in water at 25° C. and at atmospheric pressure (1.013×10⁵ Pa) (solubility of less than 5% by weight, preferably less than 1% by weight and even more preferentially less than 0.1% by weight). They bear in their structure at least one hydrocarbon-based chain including at least 6 carbon atoms and/or a sequence of at least two siloxane groups. In addition, the fatty substances are generally soluble in organic solvents under the same temperature and pressure conditions, for instance chloroform, dichloromethane, carbon tetrachloride, ethanol, benzene, toluene, tetrahydrofuran (THF), liquid petroleum jelly or decamethylcyclopentasiloxane.

In other words, the term “fatty substances” means an organic compound that is insoluble in water at 25° C. and at atmospheric pressure (1.013×10⁵ Pa), in particular with a solubility of less than 5% by weight, preferably less than 1% by weight and even more preferentially less than 0.1% by weight.

The fatty substances are different from fatty acids.

In other words, the fatty substances are particularly different from fatty acids in free form, for instance fatty acids that are not in the form of esters (or fatty acids that are not esterified).

In other words, the fatty substances are particularly different from the fatty acids previously defined.

Preferably, the fatty substances do not comprise any carboxylic acid functions —COOH or any carboxylates functions —COO⁻.

The fatty substances that may be used in the present invention are neither (poly)oxyalkylenated nor (poly)glycerolated.

In particular, the fatty substances are different from nonionic surfactants.

Preferably, the fatty substances according to the invention are neither (poly)oxyalkylenated or (poly)glycerolated and are different from nonionic surfactants.

Preferably, the fatty substances that are useful according to the invention are non-silicone fatty substances.

The term “non-silicone fatty substance” refers to a fatty substance not containing any Si—O bonds and the term “silicone fatty substance” refers to a fatty substance containing at least one Si—O bond.

Preferably, the fatty substances are non-silicone fatty substances different from nonionic surfactants and are namely neither (poly)oxyalkylenated nor (poly)glycerolated.

The fatty substances that are useful according to the invention may be liquid fatty substances (or oils) and/or solid fatty substances. A liquid fatty substance is understood to be a fatty substance having a melting point of less than or equal to 25° C. at atmospheric pressure (1.013×10⁵ Pa). A solid fatty substance is understood to be a fatty substance having a melting point of greater than 25° C. at atmospheric pressure (1.013×10⁵ Pa).

For the purposes of the present invention, the melting point corresponds to the temperature of the most endothermic peak observed on thermal analysis (differential scanning calorimetry or DSC) as described in the standard ISO 11357-3; 1999. The melting point may be measured using a differential scanning calorimeter (DSC), for example the calorimeter sold under the name MDSC 2920 by the company TA Instruments. In the present patent application, all the melting points are determined at atmospheric pressure (1.013×10⁵ Pa).

More particularly, the liquid fatty substance(s) other than fatty alcohols and fatty acids according to the invention may be chosen from C₆-C₁₆ liquid hydrocarbons, liquid hydrocarbons comprising more than 16 carbon atoms, non-silicone oils of animal origin, oils of triglyceride type of plant or synthetic origin, fluoro oils, liquid esters of fatty acid and/or of fatty alcohol other than triglycerides, and mixtures thereof.

It is recalled that the esters more particularly contain at least one saturated or unsaturated, linear or branched hydrocarbon-based group, comprising from 6 to 40 and better still from 8 to 30 carbon atoms, which is optionally substituted, in particular, with one or more (in particular 1 to 4) hydroxyl groups. If they are unsaturated, these compounds may comprise one to three conjugated or unconjugated carbon-carbon double bonds.

As regards the C₆-C₁₆ liquid hydrocarbons, the latter may be linear, branched, or optionally cyclic, and are preferably chosen from alkanes. Examples that may be mentioned include hexane, cyclohexane, undecane, dodecane, isododecane, tridecane or isoparaffins, such as isohexadecane or isodecane, and mixtures thereof.

The liquid hydrocarbons comprising more than 16 carbon atoms may be linear or branched, and of mineral or synthetic origin, and are preferably chosen from liquid paraffins or liquid petroleum jelly, polydecenes, hydrogenated polyisobutene such as Parleam®, and mixtures thereof.

A hydrocarbon-based oil of animal origin that may be mentioned is perhydrosqualene.

The triglyceride oils of plant or synthetic origin are preferably chosen from liquid fatty acid triglycerides including from 6 to 30 carbon atoms, for instance heptanoic or octanoic acid triglycerides, or alternatively, for example, sunflower oil, maize oil, soybean oil, marrow oil, grapeseed oil, sesame seed oil, hazelnut oil, apricot oil, macadamia oil, arara oil, castor oil, avocado oil, caprylic/capric acid triglycerides, for instance those sold by the company Stdarinerie Dubois or those sold under the names Miglyol® 810, 812 and 818 by the company Dynamit Nobel, jojoba oil and shea butter oil, and mixtures thereof.

As regards the fluoro oils, they may be chosen from perfluoromethylcyclopentane and perfluoro-1,3-dimethylcyclohexane, sold under the names Flutec® PCl and Flutec® PC3 by the company BNFL Fluorochemicals; perfluoro-1,2-dimethylcyclobutane; perfluoroalkanes such as dodecafluoropentane and tetradecafluorohexane, sold under the names PF 5050® and PF 5060® by the company 3M, or bromoperfluorooctyl sold under the name Foralkyl® by the company Atochem; nonafluoromethoxybutane and nonafluoroethoxyisobutane; perfluoromorpholine derivatives such as 4-trifluoromethylperfluoromorpholine sold under the name PF 5052® by the company 3M.

As regards the liquid esters of fatty acids and/or of fatty alcohols other than the triglycerides mentioned above, mention may be made notably of esters of saturated or unsaturated, linear C₁ to C₂₆ or branched C₃ to C₂₆ aliphatic monoacids or polyacids and of saturated or unsaturated, linear C₁ to C₂₆ or branched C₃ to C₂₆ aliphatic monoalcohols or polyalcohols, the total carbon number of the esters being greater than or equal to 6 and more advantageously greater than or equal to 10.

Preferably, for the esters of monoalcohols, at least one from among the alcohol and the acid is branched.

Among the monoesters, mention may be made of dihydroabietyl behenate; octyldodecyl behenate; isocetyl behenate; isostearyl lactate; lauryl lactate; linoleyl lactate; oleyl lactate; isostearyl octanoate; isocetyl octanoate; octyl octanoate; decyl oleate; isocetyl isostearate; isocetyl laurate; isocetyl stearate; isodecyl octanoate; isodecyl oleate; isononyl isononanoate; isostearyl palmitate; methyl acetyl ricinoleate; octyl isononanoate; 2-ethylhexyl isononanoate; octyldodecyl erucate; oleyl erucate; ethyl palmitate, isopropyl palmitate, 2-ethylhexyl palmitate, 2-octyldecyl palmitate, alkyl myristates such as isopropyl myristate, isobutyl stearate; 2-hexyldecyl laurate, and mixtures thereof.

Preferably, among the monoesters of monoacids and of monoalcohols, use will be made of ethyl palmitate and isopropyl palmitate, alkyl myristates such as isopropyl myristate or ethyl myristate, isocetyl stearate, 2-ethylhexyl isononanoate, isodecyl neopentanoate and isostearyl neopentanoate, and mixtures thereof.

Esters of C₄ to C₂₂ dicarboxylic or tricarboxylic acids and of C₁ to C₂₂ alcohols and esters of mono-, di- or tricarboxylic acids and of C₂ to C₂₆ di-, tri-, tetra- or pentahydroxy alcohols may also be used.

Mention may notably be made of: diethyl sebacate; diisopropyl sebacate; diisopropyl adipate; di-n-propyl adipate; dioctyl adipate; diisostearyl adipate; dioctyl maleate; octyldodecyl stearoyl stearate; pentaerythrityl monoricinoleate; pentaerythrityl tetraisononanoate; pentaerythrityl tetrapelargonate; pentaerythrityl tetraisostearate; pentaerythrityl tetraoctanoate; propylene glycol dicaprylate; propylene glycol dicaprate; tridecyl erucate; triisopropyl citrate; triisostearyl citrate; glyceryl trilactate; glyceryl trioctanoate; trioctyldodecyl citrate; trioleyl citrate; propylene glycol dioctanoate; neopentyl glycol diheptanoate; diethylene glycol diisononanoate; and polyethylene glycol distearates, and mixtures thereof.

The composition may also comprise, as fatty ester, sugar esters and diesters of C₆ to C₃₀, preferably C₁₂ to C₂₂, fatty acids. It is recalled that the term “sugar” refers to oxygen-bearing hydrocarbon-based compounds bearing several alcohol functions, with or without aldehyde or ketone functions, and which include at least 4 carbon atoms. These sugars may be monosaccharides, oligosaccharides or polysaccharides other than the anionic polysaccharides described hereinbelow.

Examples of suitable sugars that may be mentioned include sucrose, glucose, galactose, ribose, fucose, maltose, fructose, mannose, arabinose, xylose and lactose, and derivatives thereof, notably alkyl derivatives, such as methyl derivatives, for instance methylglucose.

The sugar esters of fatty acids may be chosen notably from the group comprising the esters or mixtures of esters of sugars described previously and of linear or branched, saturated or unsaturated C₆ to C₃₀ and preferably C₁₂ to C₂₂ fatty acids. If they are unsaturated, these compounds may comprise one to three conjugated or unconjugated carbon-carbon double bonds.

The esters may also be chosen from monoesters, diesters, triesters, tetraesters and polyesters, and mixtures thereof.

These esters may be, for example, oleates, laurates, palmitates, myristates, behenates, cocoates, stearates, linoleates, linolenates, caprates, arachidonates or mixtures thereof notably such as the mixed oleo-palmitate, oleo-stearate and palmitostearate esters.

More particularly, use is made of monoesters and diesters and notably sucrose, glucose or methylglucose mono- or di-oleates, -stearates, -behenates, -oleopalmitates, -linoleates, -linolenates and -oleostearates, and mixtures thereof.

An example that may be mentioned is the product sold under the name Glucate® DO by the company Amerchol, which is a methylglucose dioleate.

Preferably, use will be made of a liquid ester of a monoacid and of a monoalcohol.

The solid fatty substances preferably have a viscosity of greater than 2 Pa·s, measured at 25° C. and at a shear rate of 1 s⁻¹.

The solid fatty substance(s) other than fatty acids and fatty alcohols are preferably chosen from solid esters of fatty acids and/or of fatty alcohols, waxes, ceramides and mixtures thereof.

The solid esters of a fatty acid and/or of a fatty alcohol that may be used are preferably chosen from esters derived from a C₉-C₂₆ carboxylic fatty acid and/or from a C₉-C₂₆ fatty alcohol.

Preferably, these solid fatty esters are esters of a linear or branched, saturated carboxylic acid including at least 10 carbon atoms, preferably from 10 to 30 carbon atoms and more particularly from 12 to 24 carbon atoms, and of a linear or branched, saturated monoalcohol, including at least 10 carbon atoms, preferably from 10 to 30 carbon atoms and more particularly from 12 to 24 carbon atoms. The saturated carboxylic acids may optionally be hydroxylated, and are preferably monocarboxylic acids.

Esters of C₄-C₂₂ dicarboxylic or tricarboxylic acids and of C₁-C₂₂ alcohols and esters of mono-, di- or tricarboxylic acids and of C₂-C₂₆ di-, tri-, tetra- or pentahydroxy alcohols may also be used.

Mention may notably be made of octyldodecyl behenate, isocetyl behenate, cetyl lactate, stearyl octanoate, octyl octanoate, cetyl octanoate, decyl oleate, hexyl stearate, octyl stearate, myristyl stearate, cetyl stearate, stearyl stearate, octyl pelargonate, cetyl myristate, myristyl myristate, stearyl myristate, diethyl sebacate, diisopropyl sebacate, diisopropyl adipate, di-n-propyl adipate, dioctyl adipate, dioctyl maleate, octyl palmitate, myristyl palmitate, cetyl palmitate, stearyl palmitate, and mixtures thereof.

Preferably, the solid esters of a fatty acid and/or of a fatty alcohol are chosen from C₉-C₂₆ alkyl palmitates, notably myristyl, cetyl or stearyl palmitate; C₉-C₂₆ alkyl myristates, such as cetyl myristate, stearyl myristate and myristyl myristate; and C₉-C₂₆alkyl stearates, notably myristyl stearate, cetyl stearate and stearyl stearate; and mixtures thereof.

For the purposes of the present invention, a wax is a lipophilic compound, which is solid at 25° C. and atmospheric pressure, with a reversible solid/liquid change of state, having a melting point greater than about 40° C., which may be up to 200° C., and having in the solid state anisotropic crystal organization. In general, the size of the wax crystals is such that the crystals diffract and/or scatter light, giving the composition that comprises them a more or less opaque cloudy appearance. By bringing the wax to its melting point, it is possible to make it miscible with oils and to form a microscopically homogeneous mixture, but on returning the temperature of the mixture to room temperature, recrystallization of the wax, which is microscopically and macroscopically detectable (opalescence), is obtained.

In particular, the waxes that are suitable for use in the invention may be chosen from waxes of animal, plant or mineral origin, non-silicone synthetic waxes, and mixtures thereof.

Mention may be made notably of hydrocarbon-based waxes, for instance beeswax, notably of organic origin, lanolin wax and Chinese insect waxes; rice bran wax, carnauba wax, candelilla wax, ouricury wax, esparto grass wax, berry wax, shellac wax, Japan wax and sumac wax; montan wax, orange wax and lemon wax, microcrystalline waxes, paraffins and ozokerite; polyethylene waxes, the waxes obtained by Fischer-Tropsch synthesis and waxy copolymers, and also esters thereof.

Mention may also be made of C₂₀ to C₆₀ microcrystalline waxes, such as Microwax HW.

Mention may also be made of the MW 500 polyethylene wax sold under the reference Permalen 50-L polyethylene.

Mention may also be made of the waxes obtained by catalytic hydrogenation of animal or plant oils containing linear or branched C8 to C32 fatty chains. Among these waxes mention may notably be made of isomerized jojoba oil such as the trans-isomerized partially hydrogenated jojoba oil, notably the product manufactured or sold by the company Desert Whale under the commercial reference Iso-Jojoba-50®, hydrogenated sunflower oil, hydrogenated castor oil, hydrogenated coconut kernel oil, hydrogenated lanolin oil and bis(1,1,1-trimethylolpropane) tetrastearate, notably the product sold under the name Hest 2T-4S® by the company Heterene.

The waxes obtained by hydrogenation of castor oil esterified with cetyl alcohol, such as those sold under the names Phytowax Castor 16L64® and 22L73® by the company Sophim, may also be used.

A wax that may also be used is a C20-C40 alkyl (hydroxystearyloxy)stearate (the alkyl group containing from 20 to 40 carbon atoms), alone or as a mixture. Such a wax is notably sold under the names Kester Wax K 82 P®, Hydroxypolyester K 82 P® and Kester Wax K 80 P® by the company Koster Keunen.

It is also possible to use microwaxes in the compositions of the invention; mention may notably be made of carnauba microwaxes, such as the product sold under the name MicroCare 350® by the company Micro Powders, synthetic-wax microwaxes, such as the product sold under the name MicroEase 114S® by the company Micro Powders, microwaxes constituted of a mixture of carnauba wax and polyethylene wax, such as the products sold under the names Micro Care 300® and 310® by the company Micro Powders, microwaxes constituted of a mixture of carnauba wax and of synthetic wax, such as the product sold under the name Micro Care 325® by the company Micro Powders, polyethylene microwaxes, such as the products sold under the names Micropoly 200®, 220®, 220L® and 250S® by the company Micro Powders, and polytetrafluoroethylene microwaxes, such as the products sold under the names Microslip 519® and 519 L® by the company Micro Powders.

The waxes are preferably chosen from mineral waxes, for instance paraffin, petroleum jelly, lignite or ozokerite wax; plant waxes, for instance cocoa butter or cork fibre or sugar cane waxes, olive tree wax, rice wax, hydrogenated jojoba wax, ouricury wax, carnauba wax, candelilla wax, esparto grass wax, or absolute waxes of flowers, such as the essential wax of blackcurrant blossom sold by the company Bertin (France); waxes of animal origin, for instance beeswaxes or modified beeswaxes (cera bellina), spermaceti, lanolin wax and lanolin derivatives; microcrystalline waxes; and mixtures thereof.

The ceramides, or ceramide analogues such as glycoceramides, which may be used in the compositions according to the invention, are known; mention may be made in particular of ceramides of classes I, II, III and V according to the Dawning classification.

The ceramides or analogues thereof that may be used preferably correspond to the following formula: R³CH(OH)CH(CH₂OR²)(NHCOR¹), in which:
R¹ denotes a linear or branched, saturated or unsaturated alkyl group, derived from C₁₄-C₃₀ fatty acids, it being possible for this group to be substituted with a hydroxyl group in the alpha position, or a hydroxyl group in the omega position esterified with a saturated or unsaturated C₁₆-C₃₀ fatty acid;
R² denotes a hydrogen atom, a (glycosyl)n group, a (galactosyl)m group or a sulfogalactosyl group, in which n is an integer ranging from 1 to 4 and m is an integer ranging from 1 to 8;
R³ denotes a C₁₅-C₂₆ hydrocarbon-based group, saturated or unsaturated in the alpha position, this group possibly being substituted with one or more C₁-C₁₄ alkyl groups; it being understood that in the case of natural ceramides or glycoceramides, R³ may also denote a C₁₅-C₂₆ alpha-hydroxyalkyl group, the hydroxyl group being optionally esterified with a C₁₆-C₃₀ alpha-hydroxy acid.

The ceramides that are more particularly preferred are the compounds for which R¹ denotes a saturated or unsaturated alkyl derived from C₁₆-C₂₂ fatty acids; R² denotes a hydrogen atom and R³ denotes a saturated linear C₁₅ group.

Preferentially, use is made of ceramides for which R¹ denotes a saturated or unsaturated alkyl group derived from C₁₄-C₃₀ fatty acids; R² denotes a galactosyl or sulfogalactosyl group; and R³ denotes a —CH═CH—(CH₂)₁₂—CH₃ group.

Use may also be made of the compounds for which R¹ denotes a saturated or unsaturated alkyl radical derived from C₁₂-C₂₂ fatty acids; R² denotes a galactosyl or sulfogalactosyl radical; and R³ denotes a saturated or unsaturated C₁₂-C₂₂ hydrocarbon-based radical and preferably a —CH═CH—(CH₂)₁₂—CH₃ group.

As compounds that are particularly preferred, mention may also be made of 2-N-linoleoylaminooctadecane-1,3-diol; 2-N-oleoylaminooctadecane-1,3-diol; 2-N-palmitoylaminooctadecane-1,3-diol; 2-N-stearoylaminooctadecane-1,3-diol; 2-N-behenoylaminooctadecane-1,3-diol; 2-N-[2-hydroxypalmitoyl]aminooctadecane-1,3-diol; 2-N-stearoylaminooctadecane-1,3,4-triol and in particular N-stearoylphytosphingosine, 2-N-palmitoylaminohexadecane-1,3-diol, N-linoleoyldihydrosphingosine, N-oleoyldihydrosphingosine, N-palmitoyldihydrosphingosine, N-stearoyldihydrosphingosine, and N-behenoyldihydrosphingosine, N-docosanoyl-N-methyl-D-glucamine, cetylic acid N-(2-hydroxyethyl)-N-(3-cetyloxy-2-hydroxypropyl)amide and bis(N-hydroxyethyl-N-cetyl)malonamide; and mixtures thereof. N-Oleoyldihydrosphingosine will preferably be used.

According to a preferred embodiment, the composition according to the invention comprises at least one liquid fatty substance other than fatty alcohols and fatty acids, preferentially chosen from plant oils, liquid fatty esters, and mixtures thereof.

The total content of fatty substance(s) other than fatty alcohols and fatty acids preferably ranges from 0.1% to 20% by weight, more preferentially from 1% to 15% by weight, and better still from 2% to 10% by weight, relative to the total weight of the composition.

The total content of the solid fatty substance(s) other than fatty alcohols and fatty acids preferably ranges from 0.2% to 20% by weight, more preferentially from 1% to 15% by weight, and better still from 2% to 10% by weight, relative to the total weight of the composition.

### Surfactants

The composition according to the present invention may comprise one or more surfactants other than the fatty acids as described previously. These surfactants may be chosen from anionic surfactants other than the fatty acids as described previously, nonionic surfactants and cationic surfactants and/or mixtures thereof, preferably from anionic surfactants, nonionic surfactants and/or mixtures thereof.

Preferably, the composition according to the present invention comprises one or more surfactants.

Preferably, the surfactant(s) are chosen from anionic surfactants other than the fatty acids as described previously, nonionic and cationic surfactants, and mixtures thereof.

Preferably, the surfactant(s) are chosen from anionic surfactants other than the fatty acids as described previously, nonionic surfactants, and mixtures thereof.

The term “anionic surfactant” means a surfactant including, as ionic or ionizable groups, only anionic groups. These anionic groups are preferably chosen from the following groups: CO₂H, CO₂⁻, SO₃H, SO₃⁻, OSO₃H, OSO₃⁻, H₂PO₃, HPO₃⁻, PO₃²⁻, H₂PO₂, HPO₂⁻, PO₂²⁻, POH and PO⁻.

As examples of anionic surfactants that can be used in the composition according to the invention, mention may be made of alkyl sulfates, alkyl ether sulfates, alkylamido ether sulfates, alkylaryl polyether sulfates, monoglyceride sulfates, alkyl sulfonates, alkylamide sulfonates, alkylaryl sulfonates, α-olefin sulfonates, paraffin sulfonates, alkyl sulfosuccinates, alkyl ether sulfosuccinates, alkylamide sulfosuccinates, alkyl sulfoacetates, acyl sarcosinates, acyl glutamates, alkyl sulfosuccinamates, acyl isethionates and N—(C₁-C₄)alkyl N-acyl taurates, salts of alkyl monoesters of polyglycoside-polycarboxylic acids, acyl lactylates, salts of D-galactoside uronic acids, salts of alkyl ether carboxylic acids, salts of alkylaryl ether carboxylic acids, salts of alkylamido ether carboxylic acids; fatty acid salts, and the corresponding non-salified forms of all these compounds; the alkyl and acyl groups of all these compounds (unless specified otherwise) generally including from 6 to 24 carbon atoms and the aryl group generally denoting a phenyl group.

These compounds may be oxyethylenated and then preferably include from 1 to 50 ethylene oxide units.

The salts of C₆-C₂₄ alkyl monoesters of polyglycoside-polycarboxylic acids may be chosen from C₆-C₂₄ alkyl polyglycoside-citrates, C₆-C₂₄ alkyl polyglycoside-tartrates and C₆-C₂₄ alkyl polyglycoside-sulfosuccinates.

When the anionic surfactant(s) are in salt form, they may be chosen from alkali metal salts such as the sodium or potassium salt and preferably the sodium salt, ammonium salts, amine salts and in particular amino alcohol salts or alkaline-earth metal salts such as the magnesium salt.

Examples of amino alcohol salts that may notably be mentioned include monoethanolamine, diethanolamine and triethanolamine salts, monoisopropanolamine, diisopropanolamine or triisopropanolamine salts, 2-amino-2-methyl-1-propanol salts, 2-amino-2-methyl-1,3-propanediol salts and tris(hydroxymethyl)aminomethane salts.

Alkali metal or alkaline-earth metal salts and in particular the sodium or magnesium salts are preferably used.

The anionic surfactants that may be present may be mild anionic surfactants, i.e. anionic surfactants not bearing a sulfate function.

As regards the mild anionic surfactants, mention may be made in particular of the following compounds and salts thereof, and also mixtures thereof: polyoxyalkylenated alkyl ether carboxylic acids, polyoxyalkylenated alkylaryl ether carboxylic acids, polyoxyalkylenated alkylamido ether carboxylic acids, in particular those including 2 to 50 ethylene oxide groups, alkyl D-galactoside uronic acids, acyl sarcosinates, acyl glutamates and alkylpolyglycoside carboxylic esters.

Use may be made most particularly of polyoxyalkylenated alkyl ether carboxylic acids, for instance lauryl ether carboxylic acid (4.5 OE) sold, for example, under the name Akypo RLM 45 CA from Kao.

The nonionic surfactant(s) that may be used in the composition of the present invention are notably described, for example, in the “Handbook of Surfactants” by M.R. Porter, published by Blackie & Son (Glasgow and London), 1991, pages 116-178.

Examples of nonionic surfactants that may be mentioned include the following compounds, alone or as a mixture:
oxyalkylenated (C₈-C₂₄)alkylphenols;
saturated or unsaturated, linear or branched, oxyalkylenated or glycerolated C₈-C₄₀ alcohols, preferably including one or two fatty chains;
saturated or unsaturated, linear or branched, oxyalkylenated C₈-C₃₀ fatty acid amides;
esters of saturated or unsaturated, linear or branched, C₈-C₃₀ acids and of polyethylene glycols;
preferably oxyethylenated esters of saturated or unsaturated, linear or branched, C₈ to C₃₀ acids and of sorbitol;
esters of fatty acids, notably of C₅-C₂₄, and preferably C₁₆-C₂₂ fatty acids, and of (poly)oxyalkylenated glycerol ethers, in particular of oxyethylenated and/or oxypropylenated glycerol ethers;
fatty acid esters of sucrose;
(C₈-C₃₀)alkyl(poly)glucosides, (C₈-C₃₀)alkenyl(poly)glucosides, which are optionally oxyalkylenated (0 to 10 oxyalkylene units) and comprising from 1 to 15 glucose units, (C₈-C₃₀)alkyl(poly)glucoside esters;
saturated or unsaturated oxyethylenated plant oils;
condensates of ethylene oxide and/or of propylene oxide;
N—(C₈-C₃₀)alkylglucamine and N—(C₈-C₃₀)acylmethylglucamine derivatives;
amine oxides.

They are notably chosen from alcohols distinct from the fatty alcohols as previously described, α-diols and (C₁-C₂₀)alkylphenols, these compounds being ethoxylated, propoxylated or glycerolated and containing at least one fatty chain including, for example, from 8 to 18 carbon atoms, the number of ethylene oxide or propylene oxide groups possibly ranging notably from 1 to 200, and the number of glycerol groups possibly ranging notably from 1 to 30.

Mention may also be made of condensates of ethylene oxide and of propylene oxide with fatty alcohols, ethoxylated fatty amides preferably containing from 1 to 30 ethylene oxide units, polyglycerolated fatty amides including on average from 1 to 5, and in particular from 1.5 to 4, glycerol groups, ethoxylated fatty acid esters of sorbitan containing from 1 to 30 ethylene oxide units, fatty acid esters of sucrose, fatty acid esters of polyethylene glycol, (C₆-C₂₄ alkyl)polyglycosides, oxyethylenated plant oils, N—(C₆-C₂₄ alkyl)glucamine derivatives, amine oxides such as (C_{1O}-C₁₄ alkyl)amine oxides or N—(C₁₀-C₁₄ acyl)aminopropylmorpholine oxides.

The esters of C₈-C₃₀, notably C₈-C₂₄ and preferably C₁₆-C₂₂ fatty acids and of (poly)oxyalkylenated, in particular oxyethylenated and/or oxypropylenated, glycerol ethers, may include more than 10 oxyethylene and/or oxypropylene units, in particular from 15 to 200 units, better still from 15 to 100 oxyethylene and/or oxypropylene units.

Esters of a C₈-C₃₀ fatty acid, notably a C₈-C₂₄ and preferably C₁₆-C₂₂ fatty acid, and of (poly)oxyalkylenated glycerol ethers, in particular oxyethylenated and/or oxypropylenated glycerol ethers are preferably chosen from polyoxyethylenated glyceryl monostearate containing 200 oxyethylene units, sold under the name Simulsol 220 TM® by the company SEPPIC; polyoxyethylenated glyceryl stearate containing 30 oxyethylene units, for instance the product Tagat S® sold by the company Goldschmidt, polyoxyethylenated glyceryl oleate containing 30 oxyethylene units, for instance the product Tagat G® sold by the company Goldschmidt, polyoxyethylenated glyceryl cocoate containing 30 oxyethylene units, for instance the product Varionic LI 13® sold by the company Sherex, polyoxyethylenated glyceryl isostearate containing 30 oxyethylene units, for instance the product Tagat L® sold by the company Goldschmidt, and polyoxyethylenated glyceryl laurate containing 30 oxyethylene units, for instance the product Tagat I® from the company Goldschmidt.

The C₈-C₃₀ and preferably C₁₂-C₂₂ fatty acid esters (notably monoesters, diesters and triesters) of sorbitan may be chosen from:
sorbitan caprylate; sorbitan cocoate; sorbitan isostearate; sorbitan laurate; sorbitan oleate; sorbitan palmitate; sorbitan stearate; sorbitan diisostearate; sorbitan dioleate; sorbitan distearate; sorbitan sesquicaprylate; sorbitan sesquiisostearate; sorbitan sesquioleate; sorbitan sesquistearate; sorbitan triisostearate; sorbitan trioleate; and sorbitan tristearate.

The polyoxyethylenated C₅-C₃₀ (preferably C₁₂-C₁₈) fatty acid esters (notably monoesters, diesters and triesters) of sorbitan notably containing from 2 to 20 mol of ethylene oxide may be chosen from polyoxyethylenated esters of C₁₂-C₁₈ fatty acids, in particular lauric, myristic, cetylic or stearic acid, of sorbitan notably containing from 2 to 30 mol of ethylene oxide, such as:
polyoxyethylenated sorbitan monolaurate (4 OE) (Polysorbate-21),
polyoxyethylenated sorbitan monolaurate (20 OE) (Polysorbate-20),
polyoxyethylenated sorbitan monopalmitate (20 OE) (Polysorbate-40),
polyoxyethylenated sorbitan monostearate (20 OE) (Polysorbate-60),
polyoxyethylenated sorbitan monostearate (4 OE) (Polysorbate-61),
polyoxyethylenated sorbitan monooleate (20 OE) (Polysorbate-80),
polyoxyethylenated sorbitan monooleate (5 OE) (Polysorbate-81),
polyoxyethylenated sorbitan tristearate (20 OE) (Polysorbate-65),
polyoxyethylenated sorbitan trioleate (20 OE) (Polysorbate-85).

The polyoxyethylenated C₅-C₃₀ (preferably C₁₂-C₁₈) fatty acid esters (notably monoesters, diesters, triesters and tetraesters) of sorbitan, notably containing from 2 to 20 mol of ethylene oxide, may be chosen from polyoxyethylenated esters, notably containing from 2 to 20 mol of ethylene oxide, such as of C₁₂-C₁₈ fatty acids, in particular lauric, myristic, cetylic or stearic acid, and of sorbitan, such as:
the ester polyoxyethylenated with 20 OE of sorbitan and of cocoic acid (PEG-20 sorbitan cocoate);
the polyoxyethylenated esters (notably containing from 2 to 20 OE) of sorbitan and of isostearic acid (such as PEG-2 sorbitan isostearate; PEG-5 sorbitan isostearate; PEG-20 sorbitan isostearate such as the product sold under the name Nikkol TI 10 V by the company Nikkol),
the polyoxyethylenated esters (notably containing from 2 to 20 OE) of sorbitan and of lauric acid (such as PEG-10 sorbitan laurate),
the polyoxyethylenated esters (notably containing from 2 to 20 OE) of sorbitan and of oleic acid containing 10 oxyethylene groups (such as PEG-6 sorbitan oleate; PEG-20 sorbitan oleate),
the polyoxyethylenated esters (notably containing from 3 to 20 OE) of sorbitan and of stearic acid (such as PEG-3 sorbitan stearate; PEG-4 sorbitan stearate; PEG-6 sorbitan stearate).

The nonionic surfactant(s) are preferably chosen from ethoxylated C₈-C₂₄ fatty alcohols comprising from 1 to 200 ethylene oxide groups, ethoxylated C₈-C₃₀ fatty acid esters of sorbitan containing from 1 to 30 ethylene oxide units, (C₆-C₂₄ alkyl)polyglycosides, C₈-C₃₀, notably C₈₋₂₄, and preferably C₁₆-C₂₂ fatty acid esters of (poly)oxyalkylenated glycerol ethers, in particular of oxyethylenated and/or oxypropylenated glycerol ethers, and mixtures thereof, better still from (C₆-C₂₄ alkyl)polyglycosides, better still from (C₆-C₂₄ alkyl)polyglycosides such as: cocoyl glucoside, caprylyl/capryl glucoside, lauryl glucoside, decyl glucoside and cetearyl glucoside, and mixtures thereof.

1,4 C₈/C₁₈-alkyl(poly)glucosides, and notably whose INCI names are cocoyl glucoside and cetearyl glucoside, are most particularly preferred.

The cationic surfactant(s) that may be used in the composition according to the invention are generally chosen from optionally polyoxyalkylenated primary, secondary or tertiary fatty amines, quaternary ammonium salts, and mixtures thereof.

The fatty amines generally comprise at least one C₈-C₃₀ hydrocarbon-based chain. Among the fatty amines that may be used according to the invention, examples that may be mentioned include stearylamidopropyldimethylamine and distearylamine.

Examples of quaternary ammonium salts that may notably be mentioned include:
those corresponding to the general formula (X) below:

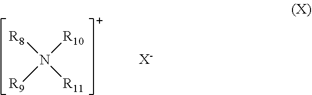

in which the groups R₈ to R₁₁, which may be identical or different, represent a linear or branched aliphatic group including from 1 to 30 carbon atoms, or an aromatic group such as aryl or alkylaryl, at least one of the groups R₈ to R₁₁ including from 8 to 30 carbon atoms and preferably from 12 to 24 carbon atoms. The aliphatic groups may include heteroatoms notably such as oxygen, nitrogen, sulfur and halogens.

The aliphatic groups are chosen, for example, from C₁-C₃₀ alkyl, C₁-C₃₀ alkoxy, polyoxy(C₂-C₆)alkylene, C₁-C₃₀ alkylamide, (C₁₂-C₂₂)alkylamido(C₂-C₆)alkyl, (C₁₂-C₂₂)alkyl acetate and C₁-C₃₀ hydroxyalkyl groups; X⁻ is an anion chosen from the group of halides, phosphates, acetates, lactates, (C₁-C₄)alkyl sulfates and (C₁-C₄)alkylsulfonates or (C₁-C₄)alkylarylsulfonates.

Among the quaternary ammonium salts of formula (X), preference is given, firstly, to tetraalkylammonium chlorides, for instance dialkyldimethylammonium or alkyltrimethylammonium chlorides in which the alkyl group includes from about 12 to 22 carbon atoms, in particular behenyltrimethylammonium chloride, distearyldimethylammonium chloride, cetyltrimethylammonium chloride or benzyldimethylstearylammonium chloride, or, secondly, to distearoylethylhydroxyethylmethylammonium methosulfate, dipalmitoylethylhydroxyethylammonium methosulfate or distearoylethylhydroxyethylammonium methosulfate, or also, finally, to palmitylamidopropyltrimethylammonium chloride or stearamidopropyldimethyl(myristyl acetate)ammonium chloride, sold under the name Ceraphyl® 70 by the company Van Dyk;
quaternary ammonium salts of imidazoline, for instance those of formula (XI) below:

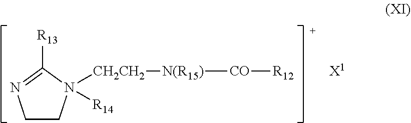

in which R12 represents an alkenyl or alkyl group including from 8 to 30 carbon atoms, for example tallow fatty acid derivatives, R13 represents a hydrogen atom, a C₁-C₄ alkyl group or an alkenyl or alkyl group including from 8 to 30 carbon atoms, R14 represents a C₁-C₄ alkyl group, R15 represents a hydrogen atom or a C₁-C₄alkyl group, and X⁻ is an anion chosen from the group of halides, phosphates, acetates, lactates, (C₁-C₄)alkyl sulfates, and (C₁-C₄)alkylsulfonates or (C₁-C₄)alkylarylsulfonates.

Preferably, R12 and R13 denote a mixture of alkenyl or alkyl groups including from 12 to 21 carbon atoms, for example tallow fatty acid derivatives, R14 denotes a methyl group and R15 denotes a hydrogen atom. Such a product is sold, for example, under the name Rewoquat® W 75 by the company Rewo,
quaternary diammonium or triammonium salts, in particular of formula (XII) below:

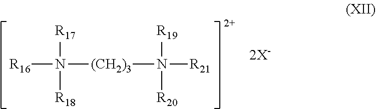

in which R16 denotes an alkyl group including approximately from 16 to 30 carbon atoms, which is optionally hydroxylated and/or interrupted with one or more oxygen atoms; R17 is chosen from hydrogen, an alkyl group including from 1 to 4 carbon atoms or a group —(CH₂)₃—N⁺ (R16a)(R17a)(R18a), R16a, R17a, R18a, R18, R19, R20 and R21, which may be identical or different, are chosen from hydrogen or an alkyl group including from 1 to 4 carbon atoms, and X⁻ is an anion chosen from the group of halides, acetates, phosphates, nitrates, (C₁-C₄)alkyl sulfates, (C₁-C₄)alkylsulfonates or (C₁-C₄)alkylarylsulfonates, in particular methyl sulfate and ethyl sulfate.

Such compounds are, for example, Finquat CT-P, sold by the company Finetex (Quaternium 89), and Finquat CT, sold by the company Finetex (Quaternium 75);
quaternary ammonium salts containing one or more ester functions, for instance those of formula (XIII) below:

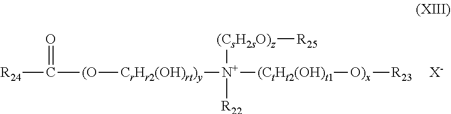

in which: R22 is chosen from C₁-C₆ alkyl groups and C₁-C₆ hydroxyalkyl or dihydroxyalkyl groups; R23 is chosen from: the group —C(O)R26, linear or branched, saturated or unsaturated C₁-C₂₂ hydrocarbon-based groups R27, or a hydrogen atom; R25 is chosen from: the group —C(O)R28, linear or branched, saturated or unsaturated C₁-C₆ hydrocarbon-based groups R29, or a hydrogen atom; R24, R26 and R28, which may be identical or different, are chosen from linear or branched, saturated or unsaturated C₇-C₂₁ hydrocarbon-based groups; r, s and t, which may be identical or different, are integers from 2 to 6; r1 and t1, which may be identical or different, are 0 or 1; r2+r1=2 r and t1+t2=2 t, y is an integer from 1 to 10, x and z, which may be identical or different, are integers from 0 to 10, X− is an organic or inorganic simple or complex anion, with the proviso that the sum x+y+z is from 1 to 15, that when x is 0, R23 denotes R27 and that when z is 0, R25 denotes R29.

The alkyl groups R22 may be linear or branched, and more particularly linear.

Preferably, R22 denotes a methyl, ethyl, hydroxyethyl or dihydroxypropyl group, and more particularly a methyl or ethyl group.

Advantageously, the sum x+y+z is from 1 to 10.

When R23 is a hydrocarbon-based group R27, it may be long and may contain 12 to 22 carbon atoms, or may be short and may contain from 1 to 3 carbon atoms.

When R25 is a hydrocarbon-based group R29, it preferably contains 1 to 3 carbon atoms.

Advantageously, R24, R26 and R28, which may be identical or different, are chosen from linear or branched, saturated or unsaturated C11-C21 hydrocarbon-based groups, and more particularly from linear or branched, saturated or unsaturated C11-C21 alkyl and alkenyl groups.

Preferably, x and z, which may be identical or different, are equal to 0 or 1.

Advantageously, y is equal to 1.

Preferably, r, s and t, which may be identical or different, are equal to 2 or 3, and even more particularly are equal to 2.

The anion X⁻ is preferably a halide, preferably chloride, bromide or iodide, a (C1-C4)alkyl sulfate or a (C1-C4)alkyl- or (C1-C4)alkylaryl-sulfonate. However, use may be made of methanesulfonate, phosphate, nitrate, tosylate, an anion derived from an organic acid, such as acetate or lactate, or any other anion that is compatible with the ammonium bearing an ester function.

The anion X⁻ is even more particularly chloride, methyl sulfate or ethyl sulfate.

Use is made more particularly, in the composition according to the invention, of the ammonium salts of formula (XIII) in which: R₂₂ denotes a methyl or ethyl group, x and y are equal to 1, z is equal to 0 or 1, r, s and t are equal to 2; R₂₃ is chosen from: the group —C(O)R₂₆, methyl, ethyl or C14-C22 hydrocarbon-based groups, or a hydrogen atom, R₂₅ is chosen from: the group —C(O)R₂₈, or a hydrogen atom, R₂₄, R₂₆ and R₂₈, which may be identical or different, are chosen from linear or branched, saturated or unsaturated C₁₃-C₁₇ hydrocarbon-based groups, and preferably from linear or branched, saturated or unsaturated C₁₃-C₁₇ alkyl and alkenyl groups.

Advantageously, the hydrocarbon-based groups are linear.

Among the compounds of formula (XIII), examples that may be mentioned include salts, notably the chloride or methyl sulfate, of diacyloxyethyldimethylammonium, diacyloxyethylhydroxyethylmethylammonium, monoacyloxyethyldihydroxyethylmethylammonium, triacyloxyethylmethyl-ammonium or monoacyloxyethylhydroxyethyldimethylammonium, and mixtures thereof. The acyl groups preferably contain 14 to 18 carbon atoms and are derived more particularly from a plant oil such as palm oil or sunflower oil. When the compound contains several acyl groups, these groups may be identical or different.

These products are obtained, for example, by direct esterification of triethanolamine, triisopropanolamine, an alkyldiethanolamine or an alkyldiisopropanolamine, which are optionally oxyalkylenated, with fatty acids or with fatty acid mixtures of plant or animal origin, or by transesterification of the methyl esters thereof. This esterification is followed by quaternization by means of an alkylating agent such as an alkyl halide, preferably methyl or ethyl halide, a dialkyl sulfate, preferably dimethyl or diethyl sulfate, methyl methanesulfonate, methyl para-toluenesulfonate, glycol chlorohydrin or glycerol chlorohydrin.

Such compounds are sold, for example, under the names Dehyquart® by the company Henkel, Stepanquat® by the company Stepan, Noxamium® by the company CECA or Rewoquat® WE 18 by the company Rewo-Witco.

The composition according to the invention may contain, for example, a mixture of quaternary ammonium monoester, diester and triester salts with a weight majority of diester salts.

Use may also be made of the ammonium salts containing at least one ester function that are described in patents U.S. Pat. Nos. 4,874,554 and 4,137,180.

Use may also be made of the behenoylhydroxypropyltrimethylammonium chloride sold, for example, by the company Kao under the name Quartamin BTC 131.

Preferably, the ammonium salts containing at least one ester function contain two ester functions.

Among the cationic surfactants, it is more particularly preferred to choose cetyltrimethylammonium, behenyltrimethylammonium and dipalmitoylethylhydroxyethylmethylammonium salts, and mixtures thereof, and more particularly behenyltrimethylammonium chloride, cetyltrimethylammonium chloride, and dipalmitoylethylhydroxyethylammonium methosulfate, and mixtures thereof.

Preferably, the surfactant(s) are chosen from nonionic surfactants and mixtures thereof, preferentially from ethoxylated C₈-C₂₄ fatty alcohols comprising from 1 to 200 ethylene oxide groups, ethoxylated C₈-C₃₀ fatty acid esters of sorbitan containing from 1 to 30 ethylene oxide units, (C₆-C₂₄ alkyl)polyglycosides, C₈-C₃₀, notably C₈-C₂₄, and preferably C₁₆-C₂₂ fatty acid esters of (poly)oxyalkylenated glycerol ethers, in particular of oxyethylenated and/or oxypropylenated glycerol ethers, and mixtures thereof.

More preferentially, the surfactant(s) are chosen from (C₆-C₂₄ alkyl)polyglycosides, esters of C₈-C₃₀, notably C₈-C₂₄ and preferably C₁₆-C₂₂ fatty acids and of (poly)oxyalkylenated glycerol ethers, in particular of oxyethylenated and/or oxypropylenated glycerol ethers, and mixtures thereof.

When the composition comprises one or more surfactants, in particular chosen from anionic and nonionic surfactants and mixtures thereof, preferably the total content of surfactant(s) in the composition ranges from 0.01% to 15% by weight, more preferentially from 0.1% to 10% by weight, better still from 0.5% to 8% by weight, even better still from 1% to 6% by weight relative to the total weight of the composition.

When the composition comprises one or more surfactants chosen from (C₆-C₂₄ alkyl)polyglycosides, esters of C₈-C₃₀, notably C₈-C₂₄ and preferably C₁₆-C₂₂ fatty acids and of (poly)oxyalkylenated glycerol ethers, in particular of oxyethylenated and/or oxypropylenated glycerol ethers, preferably the total content of surfactants in the composition ranges from 0.01% to 15% by weight, more preferentially from 0.1% to 10% by weight, better still from 0.4% to 8% by weight, even better still from 1% to 6% by weight relative to the total weight of the composition.

### Thickening Polymer

The composition according to the invention may comprise one or more thickening polymers, preferably chosen from polysaccharides, more preferentially from anionic polysaccharides.

Preferably, the composition according to the invention comprises one or more thickening polymer(s), preferably chosen from polysaccharides, more preferentially from anionic polysaccharides.

The term “polysaccharides” means polymers which contain at least 11 monosaccharide units. Preferentially, the polysaccharides of the invention include between 20 and 100 000 monosaccharide units.

The anionic polysaccharides according to the invention comprise one or more anionic or anionizable groups, and do not comprise any cationic or cationizable groups.

The anionic polysaccharides that are useful according to the invention may be chosen from those derived from the following sugars: glucose; galactose; arabinose; rhamnose; mannose; xylose; fucose; anhydrogalactose; galacturonic acid; glucuronic acid; mannuronic acid; galactose sulfate; anhydrogalactose sulfate.

The polymers bearing sugar units of the invention may be natural or synthetic.

According to a particular embodiment, the anionic polysaccharides that are useful according to the invention are chosen from native gums such as:
tree or shrub exudates, for instance: acacia gum (branched polymer of galactose, arabinose, rhamnose and glucuronic acid); ghatti gum (polymer derived from arabinose, galactose, mannose, xylose and glucuronic acid); karaya gum (polymer derived from galacturonic acid, galactose, rhamnose and glucuronic acid); gum tragacanth (polymer of galacturonic acid, galactose, fucose, xylose and arabinose);
gums derived from algae, such as: alginates (polymers of mannuronic acid and glucuronic acid); carrageenans and furcellerans (polymers of galactose sulfate and anhydrogalactose sulfate);
microbial gums such as: xanthan gums (polymer of glucose, mannose acetate, mannose/pyruvic acid and glucuronic acid); gellan gums (polymer of partially acylated glucose, rhamnose and glucuronic acid).

For the purposes of the present invention, the term “microbial gums” means substances synthesized by fermentation of sugars by microorganisms.

According to a preferred embodiment, the anionic polysaccharides that are useful according to the invention are chosen from anionic gums, better still from anionic microbial gums, more preferentially from xanthan gums.

When they are present, the total content of thickening polymers preferably ranges from 0.01% to 10% by weight relative to the total weight of the composition, preferably from 0.05% to 8% by weight, better still from 0.1% to 5% by weight relative to the total weight of the composition.

When they are present, the total content of anionic polysaccharides as defined previously preferably ranges from 0.01% to 10% by weight relative to the total weight of the composition, preferably from 0.05% to 8% by weight, better still from 0.1% to 5% by weight relative to the total weight of the composition.

The total content of the anionic microbial gums as defined previously preferably ranges from 0.01% to 10% by weight relative to the total weight of the composition, preferably from 0.05% to 5% by weight, better still from 0.1% to 2% by weight relative to the total weight of the composition.

Preferably, the composition according to the invention is an aqueous composition.

The composition preferably comprises water in an amount of greater than or equal to 5% by weight, preferably greater than or equal to 10% by weight, and better still greater than or equal to 15% by weight, relative to the total weight of the composition.

Preferably, the water content ranges from 15% to 80% by weight, preferentially from 30% to 70% by weight and better still from 40% to 60% by weight.

### Chemical Oxidizing Agents

The composition according to the invention may optionally also comprise one or more chemical oxidizing agents.

According to a particular embodiment, the composition according to the invention comprises one or more chemical oxidizing agents.

According to another particular embodiment, the composition according to the invention does not comprise any chemical oxidizing agents.

According to this embodiment, the composition according to the invention is preferably mixed at the time of use with at least one composition comprising one or more chemical oxidizing agents.

For the purposes of the present invention, the term “chemical oxidizing agent” means an oxidizing agent other than atmospheric oxygen.

The chemical oxidizing agent(s) (or decolourizing agents) that may be used in the present invention may be chosen from hydrogen peroxide, urea peroxide, alkali metal bromates, persalts such as perborates and persulfates, in particular sodium persulfate, potassium persulfate and ammonium persulfate, peracids and oxidase enzymes (with the optional cofactors thereof), among which mention may be made of peroxidases, 2-electron oxidoreductases such as uricases and 4-electron oxygenases such as laccases, and mixtures thereof; more preferentially, the chemical oxidizing agent(s) are chosen from hydrogen peroxide, persalts, and mixtures thereof, more preferably hydrogen peroxide.

Preferably, when they are present in the composition according to the invention, the chemical oxidizing agent(s) are present in a total content ranging from 0.1% to 35% by weight, more preferentially from 0.5% to 25% by weight and even more preferentially from 1% to 15% by weight, relative to the weight of the composition.

According to a preferred embodiment, when they are present in the composition according to the invention, the chemical oxidizing agent(s) chosen from hydrogen peroxide, persalts, and mixtures thereof are present in a total content ranging from 0.1% to 35% by weight, more preferentially from 0.5% to 25% by weight and even more preferentially from 1% to 15% by weight, relative to the weight of the composition.

### Additives

The composition according to the invention may contain any adjuvant or additive usually used.

Among the additives that may be contained in the composition according to the invention, mention may be made of reducing agents, thickeners, softeners, antifoams, moisturizers, UV-screening agents, peptizers, fragrances, anionic, cationic, amphoteric or zwitterionic surfactants, anionic, cationic, nonionic or amphoteric polymers or mixtures thereof, anti-dandruff agents, anti-seborrhoeic agents, vitamins and provitamins including panthenol, sunscreens, plasticizers, solubilizers, acidifying agents, mineral or organic thickeners, notably polymeric thickeners, antioxidants, hydroxy acids, fragrances, and preserving agents.

Needless to say, a person skilled in the art will take care to select this or these optional additional compounds such that the advantageous properties intrinsically associated with the composition according to the invention are not, or are not substantially, adversely affected by the envisaged addition(s).

The above additives may generally be present in an amount, for each of them, of between 0 and 20% by weight relative to the total weight of the composition.

Preferably, the composition according to the invention comprises:
one or more compounds chosen from N,N-dicarboxymethylglutamic acid, salts thereof, solvates thereof, solvates of the salts thereof and mixtures thereof,
one or more fatty alcohols chosen from unsaturated, linear liquid fatty alcohols including from 10 to 30 carbon atoms, preferably from 14 to 22 carbon atoms; saturated or unsaturated, linear or branched solid fatty alcohols including from 10 to 30 carbon atoms, preferably from 14 to 22 carbon atoms, and mixtures thereof,
one or more liquid fatty acids, preferably chosen from fatty acids including at least one carboxylic acid group and a linear or branched, unsaturated alkyl chain comprising from 10 to 30 carbon atoms, in particular from 14 to 22 carbon atoms,
one or more polyols,
one or more alkaline agents in a content of greater than or equal to 5% by weight relative to the total weight of the composition, chosen from alkanolamines, and
optionally one or more dyes, preferably chosen from oxidation dyes.

Preferably, the composition according to the invention comprises:
one or more compounds chosen from N,N-dicarboxymethylglutamic acid, salts thereof, solvates thereof, solvates of the salts thereof and mixtures thereof,
one or more fatty alcohols chosen from unsaturated, linear liquid fatty alcohols including from 14 to 22 carbon atoms; saturated or unsaturated, linear or branched solid fatty alcohols including from 14 to 22 carbon atoms, and mixtures thereof,
one or more liquid fatty acids chosen from fatty acids including at least one carboxylic acid group and a linear or branched, unsaturated alkyl chain comprising from 14 to 22 carbon atoms,
one or more polyols,
one or more alkaline agents, preferably organic alkaline agents, in a total content of greater than or equal to 5% by weight relative to the total weight of the composition, chosen from alkanolamines, and
optionally one or more dyes, preferably chosen from oxidation dyes.

Preferably, the composition according to the invention comprises:
one or more compounds chosen from N,N-dicarboxymethylglutamic acid, salts thereof, solvates thereof, solvates of the salts thereof and mixtures thereof,
one or more fatty alcohols chosen from unsaturated, linear liquid fatty alcohols including from 14 to 22 carbon atoms; saturated or unsaturated, linear or branched solid fatty alcohols including from 14 to 22 carbon atoms, and mixtures thereof,
one or more liquid fatty acids chosen from fatty acids including at least one carboxylic acid group and a linear or branched, unsaturated alkyl chain comprising from 14 to 22 carbon atoms,
one or more polyols,
one or more alkaline agents, preferably organic alkaline agents, in a total content of greater than or equal to 10% by weight relative to the total weight of the composition, chosen from alkanolamines, and
optionally one or more dyes, preferably chosen from oxidation dyes.

Preferably, the composition according to the invention comprises:
one or more compounds chosen from N,N-dicarboxymethylglutamic acid, salts thereof, solvates thereof, solvates of the salts thereof and mixtures thereof,
one or more fatty alcohols chosen from unsaturated, linear liquid fatty alcohols including from 14 to 22 carbon atoms; saturated or unsaturated, linear or branched solid fatty alcohols including from 14 to 22 carbon atoms, and mixtures thereof,
one or more liquid fatty acids chosen from fatty acids including at least one carboxylic acid group and a linear or branched, unsaturated alkyl chain comprising from 14 to 22 carbon atoms,
one or more polyols,
one or more alkaline agents, preferably chosen from alkanolamines, in a total content of greater than or equal to 10% by weight relative to the total weight of the composition, chosen from alkanolamines, and
optionally one or more dyes, preferably chosen from oxidation dyes.

### Process

The present invention also relates to a process for dyeing and/or lightening keratin fibres, in particular human keratin fibres such as the hair, in which a composition as defined previously is applied to said fibres, comprising:
one or more compounds chosen from N,N-dicarboxymethylglutamic acid, salts thereof, solvates thereof, solvates of the salts thereof and mixtures thereof,
one or more fatty alcohols,
one or more fatty acids,
one or more polyols,
one or more alkaline agents in a total content of greater than or equal to 5% by weight relative to the total weight of the composition, and
optionally one or more dyes.

Preferably, the present invention also relates to a process for dyeing keratin fibres, in particular human keratin fibres such as the hair, in which a composition as defined previously is applied to said fibres, comprising:
one or more compounds chosen from N,N-dicarboxymethylglutamic acid, salts thereof, solvates thereof, solvates of the salts thereof and mixtures thereof,
one or more fatty alcohols,
one or more fatty acids,
one or more polyols,
one or more alkaline agents in a total content of greater than or equal to 5% by weight relative to the total weight of the composition, and
one or more dyes.

In other words, the dyeing process comprises at least the application of a composition according to the invention optionally comprising one or more dyes. The dye(s) may be chosen from oxidation dyes, direct dyes and mixtures thereof, preferentially chosen from oxidation dyes.

Preferably, the composition according to the invention comprises one or more fatty substances, as defined previously, other than fatty alcohols and fatty acids.

In particular, the dyeing process comprises at least one step of applying a composition as defined previously.

Preferably, the composition according to the invention is a composition for dyeing keratin fibres, such as the hair. Thus, preferably, the composition according to the invention comprises one or more dyes chosen from oxidation dyes, direct dyes and mixtures thereof as described previously, preferably from oxidation dyes.

Preferably, the dyeing process comprises at least the application to said keratin fibres of at least one composition according to the invention comprising one or more dyes chosen from oxidation dyes, direct dyes and mixtures thereof, preferably from oxidation dyes, in the presence of one or more chemical oxidizing agents, as defined previously, preferably hydrogen peroxide.

Preferably, the dyeing and/or lightening process comprises at least the application to said fibres of a composition according to the invention comprising:
one or more compounds chosen from N,N-dicarboxymethylglutamic acid, salts thereof, solvates thereof, solvates of the salts thereof and mixtures thereof,
one or more fatty alcohols,
one or more fatty acids,
one or more polyols,
one or more alkaline agents in a total content of greater than or equal to 5% by weight relative to the total weight of the composition,
optionally one or more dyes, preferably chosen from oxidation dyes,
one or more chemical oxidizing agents as defined previously, preferably hydrogen peroxide.

Preferably, the dyeing process comprises at least the application to said fibres of a composition according to the invention comprising:
one or more compounds chosen from N,N-dicarboxymethylglutamic acid, salts thereof, solvates thereof, solvates of the salts thereof and mixtures thereof,
one or more fatty alcohols,
one or more fatty acids,
one or more polyols,
one or more alkaline agents in a total content of greater than or equal to 5% by weight relative to the total weight of the composition,
one or more dyes, preferably chosen from oxidation dyes,
one or more oxidizing agents as defined previously, preferably hydrogen peroxide.

According to a particular embodiment, the process comprises at least the use of a composition according to the invention and at least the use of a composition comprising one or more chemical oxidizing agents as defined previously, preferably hydrogen peroxide.

According to a particular embodiment, the dyeing and/or lightening process comprises at least the application to said fibres of a composition obtained by mixing, at the time of use:
at least one composition, as defined above, comprising:
one or more compounds chosen from N,N-dicarboxymethylglutamic acid, salts thereof, solvates thereof, solvates of the salts thereof and mixtures thereof,
one or more fatty alcohols,
one or more fatty acids,
one or more polyols,
one or more alkaline agents in a content of greater than or equal to 5% by weight relative to the total weight of the composition,
optionally one or more dyes, preferably chosen from oxidation dyes,
   at least one composition comprising one or more chemical oxidizing agents as described previously, preferably hydrogen peroxide.

According to a particular embodiment, the dyeing process comprises at least the application to said fibres of a composition obtained by mixing at the time of use:
at least one composition, as defined above, comprising:
one or more compounds chosen from N,N-dicarboxymethylglutamic acid, salts thereof, solvates thereof, solvates of the salts thereof and mixtures thereof,
one or more fatty alcohols,
one or more fatty acids,
one or more polyols,
one or more alkaline agents in a content of greater than or equal to 5% by weight relative to the total weight of the composition,
one or more dyes, preferably chosen from oxidation dyes,
   at least one composition comprising one or more chemical oxidizing agents as described previously, preferably hydrogen peroxide.

The oxidizing composition is preferably an aqueous composition. In particular, it comprises more than 5% by weight of water, preferably more than 10% by weight of water and even more advantageously more than 20% by weight of water.

The oxidizing composition also preferably comprises one or more acidifying agents. Among the acidifying agents, examples that may be mentioned include mineral or organic acids, for instance hydrochloric acid, orthophosphoric acid, sulfuric acid, carboxylic acids, for instance acetic acid, tartaric acid, citric acid or lactic acid, and sulfonic acids.

The oxidizing composition may also comprise fatty substances such as those described previously, preferably chosen from fatty alcohols, liquid hydrocarbons comprising more than 16 carbon atoms and mixtures thereof, surfactants and polymers.

Usually, the pH of the oxidizing composition, when it is aqueous, is less than 7.

Preferably, the oxidizing composition comprises hydrogen peroxide as oxidizing agent, in aqueous solution, the concentration of which ranges, more particularly, from 0.1% to 30%, more particularly between 1% and 20% and even more preferentially between 2% and 15% by weight, relative to the weight of the oxidizing composition.

The composition according to the invention may be used on wet or dry keratin fibres, and also on any type of fair or dark, natural or dyed, permanent-waved, bleached or relaxed fibres.

According to a particular embodiment of the process of the invention, the fibres are washed before applying the composition described above.

The application of the composition of the invention to the keratin fibres may be performed via any conventional means, in particular by means of a comb, a fine brush, a coarse brush or with the fingers.

The application of the composition according to the invention to the keratin fibres is generally performed at room temperature (between 15 and 25° C.).

The composition according to the invention may be applied to the keratin fibres for a leave-on time ranging from 30 to 60 minutes.

After applying the composition according to the invention, the keratin fibres may optionally be washed with a shampoo and/or rinsed with water.

The present invention also relates to the use of the composition according to the invention as described previously for dyeing and/or lightening keratin fibres, preferably dyeing keratin fibres, in particular human keratin fibres such as the hair.

The present invention also relates to a ready-to-use composition, as defined previously, comprising:
one or more compounds chosen from N,N-dicarboxymethylglutamic acid, salts thereof, solvates thereof, solvates of the salts thereof and mixtures thereof,
one or more fatty alcohols, as defined previously,
one or more fatty acids, as defined previously,
one or more polyols, as defined previously,
one or more alkaline agents, as defined previously, in a total content of greater than or equal to 5% by weight relative to the total weight of the composition,
optionally one or more dyes, as defined previously, and
one or more chemical oxidizing agents as defined previously, preferably hydrogen peroxide.

The invention also relates to a multi-compartment device comprising at least a first compartment containing the composition according to the invention as described above, in particular also comprising one or more dyes chosen from oxidation dyes, direct dyes and mixtures thereof, and at least a second compartment containing one or more chemical oxidizing agents as described above, preferably hydrogen peroxide.

The examples that follow serve to illustrate the invention without, however, being limiting in nature.

### EXAMPLES

### Example 1

Composition A1 (invention) and composition B1 (comparative) were prepared from the following ingredients, expressed as weight percentages:

TABLE 1

| | Composition B1 | Composition A1 |
|---|---|---|
| Xanthan gum | 0.2 | 0.2 |
| Oleic acid | 2.7 | 2.7 |
| Ethanolamine | 3.13 | 13.13 |
| Cocoyl glucoside | 1.87 | 1.87 |
| 1,3-Propanediol | 5 | 5 |
| Tetrasodium glutamate diacetate | 0.24 | 0.24 |
| Glycerol | 5 | 5 |
| Cetearyl alcohol | 16.3 | 16.3 |
| Oleyl alcohol | 2.7 | 2.7 |
| Water | qs 100 | qs 100 |

Composition C1 was prepared from the ingredients below, expressed as weight percentages:

TABLE 2

| | Composition C1 |
|---|---|
| Trideceth-2 Carboxamide MEA | 0.85 |
| Tetrasodium etidronate | 0.06 |
| Sodium salicylate | 0.035 |
| Glycerol | 0.50 |
| Cetearyl alcohol | 2.28 |
| Ceteareth-25 | 0.57 |
| Phosphoric acid | qs pH = 2.2 ± 0.2 |
| Hydrogen peroxide | 6 |
| Tetrasodium pyrophosphate | 0.04 |
| Water | qs 100 |

At the time of use, each of the compositions A1 and B1 is mixed with 1.5 times the amount of composition C1.

Each of the mixtures is then applied to a lock of chestnut-brown hair (tone depth of 4), at a rate of 5 g of mixture per gram of hair.

After a leave-on time of 30 minutes on a plate thermostatically regulated at 27° C., the hair is rinsed, washed with Pro Classique L'Oreal Professionnel shampoo and dried.

### Results—Lightening

The lightening of the hair is evaluated in the L*a*b* system, using a Konica Minolta CM-3600A spectrocolorimeter (illuminant D65, angle 10°, specular component included) in the CIELab system.

In this system, L* represents the lightness of the lightening. The greater the value of L*, the more the colouring obtained is lightened. The chromaticity is measured by the values a* and b*, a* representing the red/green axis and b* the yellow/blue axis.

The results are shown in the table below:

TABLE 3

| | L* |
|---|---|
| Mixture B1 + C1 (comparative) | 20.8 |
| Mixture A1 + C1 (invention) | 25 |

The composition according to the invention leads to better lightening performance, in comparison with the comparative composition.

### Example 2

Composition A2 (invention) and composition B2 (comparative) were prepared from the following ingredients, expressed in % by weight:

TABLE 4

| | A2 | B2 |
|---|---|---|
| | Invention | Comparative |
| Xanthan gum | 0.2 | 0.2 |
| Oleic acid | 2.7 | 2.7 |
| Ethanolamine | 12.35 | 12.35 |
| 2-amino-3-hydroxypyridine | 0.01 | 0.01 |
| 6-hydroxyindole | 0.13 | 0.13 |
| Hydroxybenzomorpholine | 1.14 | 1.14 |
| 1,3-propanediol | 5 | 5 |
| Ascorbic acid | 0.25 | 0.25 |
| Tetrasodium glutamate diacetate | 0.2 | — |
| EDTA | — | 0.2 |
| N,N-Bis(2-hydroxyethyl)-p-phenylenediamine sulfate | 0.16 | 0.16 |
| Cetearyl glucoside | 0.5 | 0.5 |
| Glycerin | 5 | 5 |
| m-Aminophenol | 0.38 | 0.38 |
| 2,4-Diaminophenoxyethanol HCL | 0.05 | 0.05 |
| Cetearyl alcohol | 16.3 | 16.3 |
| Oleyl alcohol | 2.7 | 2.7 |
| Sodium metabisulfite | 0.71 | 0.71 |
| Hydroxyethyl-3,4-methylenedioxyaniline HCL | 0.23 | 0.23 |
| Toluene-2,5-diamine | 1.55 | 1.55 |
| Water | Qs 100 | Qs 100 |

Composition C2 was prepared from the following ingredients, expressed in % by weight:

TABLE 5

| | C2 |
|---|---|
| Trideceth-2 Carboxamide MEA | 0.85 |
| Tetrasodium etidronate | 0.06 |
| Sodium salicylate | 0.035 |
| Glycerin | 0.5 |
| Cetearyl alcohol | 2.28 |
| Ceteareth-25 | 0.57 |
| Phosphoric acid | Qs pH = 2.2 +/− 0.2 |
| Hydrogen peroxide | 6 |
| Tetrasodium pyrophosphate | 0.04 |
| Water | Qs 100 |

At the moment of use, each of the compositions A2 and B2 is mixed with one and a half times of oxidizing composition C2.

Each mixture is then applied to locks of natural hair containing 90% white hairs (WN) and permanent-waved hair (PW). The “mixture/lock” bath ratio is, respectively 10/1 (g/g), i.e. 10 grams of mixture for 1 gram of hair.

After a leave-on-time period of 30 minutes on a plate thermostatically regulated at 27° C., the locks are rinsed, and then washed with a standard shampoo. The locks are dried afterwards.

### Selectivity:

The color of locks is assessed in the CIE L*a*b* system, using a spectrocolorimeter SpectraMagic NX Konica Minolta (illuminant D65, angle 10°, specular component included).

The selectivity coloration AE corresponds to the variation in colour between the lock of colored natural white hair (WN), representative of the nature of the hair at the root, and the lock of colored permanent-waved hair (PW) which is representative of the nature of the hair at the tip.

The lower the value of ΔE, the lower the selectivity coloration and therefore the more uniform the coloration between the tip and the root of the hair.

The selectivity coloration ΔE is calculated from the values of L*, a* and b* measured according to the following equation (i):

$$Δ E = \sqrt{{( {L}^{★} - {L}_{◦}^{★} )}^{2} + {( {a}^{★} - {a}_{◦}^{★} )}^{2} + {( {b}^{★} - {b}_{◦}^{★} )}^{2}} \qquad ( i )$$

In equation (i), L*, a* and b* represent the values measured on locks of dyed natural white hair (WN) and L₀*, a₀* and b₀* represent the values measured on locks of dyed permanent-waved hair (PW).

### Results:

The results are given in the table below:

TABLE 6

| | Lock type | L* | a* | b* | ΔE |
|---|---|---|---|---|---|
| A2 + C2 | WN | 19.37 | 1.53 | 1.11 | 1.23 |
| Invention | WP | 18.31 | 1.61 | 1.73 | |
| B2 + C2 | WN | 22.5 | 1.6 | 2.23 | 3.71 |
| Comparative | WP | 18.81 | 1.66 | 1.85 | |

Table 6 shows that the composition according to the present invention, i.e. comprising GLDA, leads to a significant lower ΔE value, therefore to less selective colorations, than the comparative composition comprising EDTA.

Hence the composition according to the present invention outperforms the comparative composition in terms of selectivity.

### Example 3

The dye compositions A3 was prepared from the ingredients of which the contents are indicated in the table below (% AM):

### Dye Composition (A3)

TABLE 7

| | A3 |
|---|---|
| XANTHAN GUM | 0.2 |
| OLEIC ACID | 2.7 |
| ETHANOLAMINE | 12.7 |
| COCO-GLUCOSIDE | 1.87 |
| HYDROXYETHYL-3,4- | 0.3 |
| METHYLENEDIOXYANILINE HCL | |
| HYDROXYPROPYL-P- | 0.3 |
| PHENYLENEDIAMINE HCL | |
| 1,3-PROPANEDIOL | 5 |
| TETRASODIUM GLUTAMATE DIACETATE | 0.24 |
| GLYCERIN | 5 |
| CETEARYL ALCOHOL | 16.3 |
| OLEYL ALCOHOL | 2.7 |
| WATER | Qs 100 |

At the time of use, composition A3 is mixed with 1.5 times its weight of oxidizing agent C1 disclosed in example 1.

The mixtures is then applied to locks of 90% permanent-waved white hair, in a proportion of 5 g of mixture/g of hair.

After a leave-on time of 30 minutes on a plate thermostatically regulated at 27° C., the hair is rinsed, washed with a standard shampoo and dried.

### Results

The colorimetric measurements were performed using a Konica Minolta CM-3600A spectrocolorimeter (illuminant D65, angle 10°, specular component included) in the CIELab system.

The chromaticity is calculated by the following formula:

$${C}^{*} = \sqrt{( {a}^{*} ) + {( {b}^{*} )}^{2}}$$

a* indicates the green/red color axis
b* indicates the blue/yellow color axis

The higher the value of the chromaticity C*, the greater the chromaticity is.

The composition according to the invention leads to a high C* value, and thus to a good chromaticity.

1. Composition comprising:
one or more compounds chosen from N,N-dicarboxymethylglutamic acid, salts thereof, solvates thereof, solvates of the salts thereof and mixtures thereof,
one or more fatty alcohols,
one or more fatty acids,
one or more polyols,
one or more alkaline agents in a total content of greater than or equal to 5% by weight relative to the total weight of the composition,
optionally one or more dyes.

2. Composition according to claim 1, characterized in that the compound(s) chosen from N,N-dicarboxymethylglutamic acid, salts thereof, solvates thereof, solvates of the salts thereof and mixtures thereof are chosen from alkali metal salts, preferably tetrasodium glutamate diacetate.

3. Composition according to claim 1 or 2, characterized in that the total content of the compound(s) chosen from N,N-dicarboxymethylglutamic acid, salts thereof, solvates thereof, solvates of the salts thereof and mixtures thereof ranges from 0.001% to 15% by weight, more preferentially from 0.005% to 10% by weight, better still from 0.01% to 8% by weight, even better still from 0.05% to 5% by weight, or even from 0.075% to 2% by weight, or even from 0.1% to 1% by weight, relative to the total weight of the composition.

4. Composition according to any one of the preceding claims, characterized in that the fatty alcohols are chosen from alcohols comprising 14 to 40 carbon atoms, preferably from fatty alcohols comprising 14 to 22 carbon atoms, more preferentially from cetyl alcohol, stearyl alcohol, cetearyl alcohol, oleyl alcohol, and mixtures thereof.

5. Composition according to any one of the preceding claims, characterized in that the fatty acids include at least one carboxylic acid and a linear or branched, saturated or unsaturated, in particular unsaturated, alkyl chain comprising from 10 to 30 carbon atoms, in particular from 14 to 22 carbon atoms.

6. Composition according to any one of the preceding claims, characterized in that the fatty acids are liquid fatty acids, in particular chosen from oleic acid, linoleic acid, linolenic acid and mixtures thereof, more preferentially oleic acid.

7. Composition according to any one of the preceding claims, characterized in that the polyols are chosen from diglycerol, glycerol, propylene glycol, propane-1,3-diol, 1,3-butylene glycol, pentane-1,2-diol, octane-1,2-diol, dipropylene glycol, hexylene glycol, ethylene glycol, polyethylene glycols, sorbitol, sugars such as glucose, and mixtures thereof, preferably from glycerol, propane-1,3-diol and mixtures thereof.

8. Composition according to any one of the preceding claims, characterized in that the alkaline agents are chosen from alkanolamines such as monoethanolamine, diethanolamine or triethanolamine; aqueous ammonia, carbonates or bicarbonates such as sodium (hydrogen) carbonate and potassium (hydrogen) carbonate, alkali metal or alkaline-earth metal silicates or metasilicates such as sodium metasilicate and mixtures thereof; more preferentially from aqueous ammonia and alkanolamines, better still from alkanolamines, and better still the alkaline agent is monoethanolamine.

9. Composition according to any one of the preceding claims, characterized in that the total content of alkaline agents ranges from 5% to 40% by weight, more preferentially from 6% to 30% by weight, better still from 8% to 20% by weight and even better still from 10% to 15% by weight relative to the total weight of the composition.

10. Composition according to any one of the preceding claims, characterized in that it comprises one or more dyes chosen from oxidation dyes, direct dyes and mixtures thereof, preferably oxidation dyes chosen from oxidation bases optionally associated with one or more couplers.

11. Composition according to any one of the preceding claims, characterized in that it comprises one or more chemical oxidizing agents, preferably chosen from hydrogen peroxide, urea peroxide, alkali metal bromates, persalts such as perborates and persulfates, peracids and oxidase enzymes (with the possible cofactors thereof), and mixtures thereof; preferably, the chemical oxidizing agent(s) are chosen from hydrogen peroxide, persalts, and mixtures thereof, more preferentially hydrogen peroxide.

12. Process for dyeing and/or lightening keratin fibres, in particular human keratin fibres such as the hair, comprising the application to said fibres of a composition as defined according to any one of claims 1 to 11.

13. Process according to claim 12, characterized in that it comprises at least the application to said fibres of a composition obtained by mixing, at the time of use:
at least one composition as defined according to any one of claims 1 to 10,
at least one composition comprising one or more chemical oxidizing agents as defined according to claim 11.

14. Use of a composition according to any one of claims 1 to 11, for dyeing and/or lightening keratin fibres, in particular human keratin fibres such as the hair.

15. Multi-compartment device comprising at least a first compartment containing the composition as defined according to any one of claims 1 to 10 and at least a second compartment containing a composition comprising one or more chemical oxidizing agents as defined according to claim 11.
